(19) United States
(12) Patent Application Publication
(10) Pub. No.: US 2005/0266548 A1
(43) Pub. Date: Dec. 1, 2005

(54) Biocatalyst chamber encapsulation system for bioremediation and fermentation with improved rotor

(75) Inventors: Heath H. Herman, Tucker, GA (US); Firoz R. Mistry, Chapel Hill, NC (US); Tod M. Herman, Hillsborough, NC (US)

Correspondence Address: TROUTMAN SANDERS LLP, BANK OF AMERICA PLAZA, SUITE 5200, 600 PEACHTREE STREET , NE, ATLANTA, GA, 30308-2216, US

(73) Assignee: KBI BioPharma, Inc., Durham, NC (US)

(21) Appl. No.: 11/178,556

(22) Filed: Jul. 11, 2005

Related U.S. Application Data
(63) Continuation-in-part of application No. 10/153,161, filed on May 22, 2002, now Pat. No. 6,916,652.
(63) Continuation-in-part of application No. 09/788,991, filed on Feb. 20, 2001, now abandoned.
Application No. 09/788,991 is a continuation-in-part of application No. 09/316,566, filed on May 21, 1999, now Pat. No. 6,660,509.
Application No. 09/316,566 is a continuation-in-part of application No. 09/224,645, filed on Dec. 31, 1998, now Pat. No. 6,214,617.
Application No. 09/224,645 is a continuation-in-part of application No. 09/115,109, filed on Jul. 13, 1998, now Pat. No. 6,133,019.
Application No. 09/115,109 is a continuation-in-part of application No. 08/784,718, filed on Jan. 16, 1997, now Pat. No. 5,821,116.
Application No. 08/784,718 is a division of application No. 08/412,289, filed on Mar. 28, 1995, now Pat. No. 5,622,819.
(60) Provisional application No. 60/292,755, filed on May 22, 2001.

(51) Int. Cl. C12M 1/14

(52) U.S. Cl. 435/289.1; 435/299.1

(57) ABSTRACT

The invention comprises novel culture methods and devices in which biocatalysts are substantially immobilized contained, suspended and/or incubated in a chamber. At least one injection element provides a fluid flow to a perimeter of the chamber and the fluid force of the fluids flowing into the chamber works with the centripetal force created by rotation of the device to suspend the cells within the chamber, promote cell growth and/or clean the fluid as it passes thru the suspended biocatalyst. Other embodiments are also claimed and described.

### CROSS-REFERENCE TO RELATED APPLICATIONS

This application is a continuation-in-part of U.S. patent application Ser. No. 10/153,161, filed May 22, 2002, now U.S. Pat. No. 6,916,652, which claims priority to U.S. Provisional Patent App. Ser. No. 60/292,755, filed May 22, 2001; and is a continuation-in-part of U.S. patent application Ser. No. 09/788,991, filed Feb. 20, 2001, now abandoned; which claims priority to U.S. Provisional Patent App. Ser. No. 60/179,273, filed Jan. 31, 2000, and is a continuation-in-part of U.S. Pat. No. 6,660,509 (application Ser. No. 09/316,566, filed 21 May 1999); which is a continuation-in-part of U.S. Pat. No. 6,214,617 (application Ser. No. 09/224,645, filed 31 Dec. 1998); which is a continuation-in-part of U.S. Pat. No. 6,133,019 (application Ser. No. 09/115,109, filed 13 Jul. 1998); which is a continuation-in-part of U.S. Pat. No. 5,821,116 (application Ser. No. 08/784,718, filed 16 Jan. 1997); which is a division of U.S. Pat. No. 5,622,819 (application Ser. No. 08/412,289, filed 28 Mar. 1995). Each of the above listed patent applications and patents are incorporated herein by reference as if fully set forth below.

### FIELD OF THE INVENTION

The invention relates to an improved method and apparatus for the continuous culture of biocatalysts. More particularly, the invention relates to a method and apparatus for culturing micro-organisms, or plant or animal cells, or subcellular cell components as three-dimensional arrays immobilized in centrifugal force fields which are opposed by liquid flows. The invention allows the maintenance of extremely high density cultures of biocatalysts and maximizes their productivity.

### BACKGROUND OF THE INVENTION

The term “fermentation” as used herein means any of a group of chemical reactions induced by living or nonliving biocatalysts. The term “culture” as used herein means the suspension or attachment of any such biocatalyst in or covered by a liquid medium for the purpose of maintaining chemical reactions. The term “biocatalysts” as used herein, includes enzymes, vitamins, enzyme aggregates, immobilized enzymes, subcellular components, prokaryotic cells, and eukaryotic cells. The term “centrifugal force” means a centripetal force resulting from angular rotation of an object when viewed from a congruently rotating frame of reference.

The culture of microbial cells (fermentation) or animal and plant cells (tissue culture) are central to a multiplicity of commercially-important chemical and biochemical production processes. Living cells are employed in these processes as a result of the fact that living cells, using generally easily obtainable starting materials, can economically synthesize commercially-valuable chemicals.

Fermentation involves the growth or maintenance of living cells in a nutrient liquid media. In a typical batch fermentation process, the desired micro-organism or eukaryotic cell is placed in a defined medium composed of water, nutrient chemicals and dissolved gases, and allowed to grow (or multiply) to a desired culture density. The liquid medium must contain all the chemicals which the cells require for their life processes and also should provide the optimal environmental conditions for their continued growth and/or replication. Currently, a representative microbial cell culture process might utilize either a continuous stirred-tank reactor or a gas-fluidized bed reactor in which the microbe population is suspended in circulating nutrient media. Similarly, in vitro mammalian cell culture might employ a suspended culture of cells in roller flasks or, for cells requiring surface attachment, cultures grown to confluence in tissue culture flasks containing nutrient medium above the attached cells. The living cells, so maintained, then metabolically produce the desired product(s) from precursor chemicals introduced into the nutrient mixture. The desired product(s) are either purified from the liquid medium or are extracted from the cells themselves.

Examples of methods employing fermentations of cells growing in either agitated aqueous suspension or with surface attachment are described, for example, in U.S. Pat. Nos. 3,450,598; 3,843,454; 4,059,485; 4,166,768; 4,178,209; 4,184,916; 4,413,058; and 4,463,019. Further reference to these and other such conventional cell culturing techniques may be found in such standard texts as Kruse and Patterson, Tissue Culture Methods and Applications, Academic Press, New York, 1977; and Collins and Lyne's Microbiological Methods, Butterworths, Boston, 1989.

There are a number of disadvantages inherent in such typical fermentation processes. On a commercial scale, such processes require expensive energy expenditures to maintain the large volumes of aqueous solution at the proper temperature for optimal cell viability. In addition, because the metabolic activity of the growing cell population causes decreases in the optimal levels of nutrients in the culture media and causes changes in the media pH, the process must be continuously monitored and additions must be made to maintain nutrient concentration and pH at optimal levels.

In addition, the optimal conditions under which the desired cell type may be cultured are usually near the optimal conditions for the growth of many other undesirable cells or microorganisms. Extreme care and expense must be taken to initially sterilize and to subsequently exclude undesired cell types from gaining access to the culture medium. Next, such fermentation methods, particularly those employing aerobic organisms, are quite often limited to low yields of product or low rates of product formation as a result of the inability to deliver adequate quantities of dissolved oxygen to the metabolizing organism. Finally, such batch or semi-batch processes can only be operated for a finite time period before the buildup of excreted wastes in the fermentation media require process shutdown followed by system cleanup, resterilization, and a re-start.

The high costs associated with the preparation, sterilization, and temperature control of the large volumes of aqueous nutrient media needed for such cultures has led to the development of a number of processes whereby the desired cell type or enzyme can be immobilized in a much smaller volume through which smaller quantities of nutrient media can be passed. Cell immobilization also allows for a much greater effective density of cell growth and results in a much reduced loss of productive cells to output product streams. Thus, methods and processes for the immobilization of living cells are of considerable interest in the development of commercially valuable biotechnologies.

Accordingly, there remains a need for an apparatus and method for continuously culturing, feeding, and extracting biochemical products from either microbial or eukaryotic cells or their subcellular components while maintaining viable, high density aggregates of these biocatalysts. In addition, there is a need for a method for the absolute immobilization of sample biocatalyst populations which will allow the study of various nutritive, growth, and productive parameters to provide a more accurate understanding of the inter-relationships between these parameters and their effects on cell viability and productivity.

### BRIEF SUMMARY OF THE INVENTION

An embodiment of the invention comprises a novel culture method and apparatus in which living cells or subcellular biocatalysts are immobilized within bioreactor chambers mounted in a centrifugal field while nutrient liquids, without any gas phase(s) in contact with the liquids, are flowed into and out of the bioreactor chambers. The cells or biocatalysts are ordered into a three-dimensional array of particles, the density of which is determined by the particle size, shape, intrinsic density, and by the selection of combinations of easily controllable parameters such as liquid flow rate and angular velocity of rotation.

According to an embodiment of the invention, the cells or biocatalysts can be confined within the bioreactor chambers at a defined volume. Only liquids (which may contain dissolved gases) are passed into and out of the bioreactor chambers. To cause nutrient liquids to flow through the three-dimensional array of cells or catalysts in the bioreactor chambers, positive displacement pumps are employed to move the nutrient liquid, at positive hydraulic pressure, through the bioreactor chambers. The confined cells or biocatalysts are unaffected by the resultant increase in hydraulic pressure as long as high-frequency pressure fluctuations are not present. Thus, fresh, optimal liquid nutrient media is presented to the confined cells or biocatalysts at all times during the process flow while desired cellular products are immediately accessible at the output of the bioreactor chambers.

In an alternative embodiment of the invention, the living cells or subcellular biocatalysts are not confined in closed bioreactor chambers, but rather are immobilized in open chambers formed by and between adjacent disks. As with other disclosed embodiments of the invention, the inflow of nutrient fluid into the chamber counterbalances the centrifugal force exerted on the cells to immobilize the cells in the open chamber. Use of an open chamber, however, greatly increases the capacity of the device to produce the desired cellular products.

An embodiment of the invention can be used to produce high yields of industrial chemicals or pharmaceutical products from biocatalysts such as bacteria, yeasts, fungi, and eukaryotic cells or subcellular organelles, such as mitochondria, or immobilized enzyme complexes. These cells or cellular substructures can be either naturally occurring or can be genetically manipulated to produce the desired product. These embodiments of the invention can be operated in either of two modes: (1) a mode in which nutrient limitation is used to ensure a defined bioreactor bed volume. This mode is applicable to cultures where desired products are released from the immobilized biocatalysts and exit the bioreactor in the liquid flow; (2) a mode in which excess nutrient input is used to cause overgrowth of the volume limitation of the bioreactor. This mode is useful for the continual production and outflow of mature cells containing an intracellular product.

Accordingly, it is an object of the invention to provide a method and apparatus by which biocatalysts are immobilized within bioreactor chambers while nutrient liquids are fed into the bioreactor chambers and effluent liquids containing desired metabolic product(s) exit the bioreactor chambers.

It is a further object of the invention to provide a method and apparatus by which biocatalysts, including living cell populations, may be immobilized and either aerobic or anaerobic fermentations performed in which liquid nutrient and substrate nutrients are converted to product-containing output liquid streams.

These and other objects, features and advantages of the invention will become apparent after a review of the following detailed description of the disclosed embodiment and the appended claims.

### BRIEF DESCRIPTION OF DRAWINGS

The accompanying figures, which are incorporated and form a part of the specification, illustrate several scientific principles and embodiments of the invention and, together with the description, serve to explain the principles of the invention.

FIG. 1 illustrates a central problem with Counter-Flow Centrifugation.

FIG. 2 illustrates a mathematical defect in the conventional treatment of Counter-Flow Centrifugation.

FIG. 3 is an illustration of the effect on immobilized particles using conventional Counter-Flow Centrifugation at long time periods.

FIG. 4 illustrates a modification of Counter-Flow Centrifugation in accordance with some embodiments of the present invention.

FIG. 5 is an illustration of the mathematics governing the motion of a particle due to the effect of gravity on that particle when it is restrained in a centrifugal field exactly opposed by a liquid flow.

FIG. 6 is an illustration of the resultant motion of a particle under the constraints of FIG. 5.

FIG. 7 is a mathematical evaluation of the immobilization conditions at a given radius.

FIG. 8 is a block diagram of a process configuration designed to maintain desired dissolved gas concentrations in the liquid input to a centrifugal bioreactor.

FIG. 9 is an illustration of a representative liquid flow pressure regulator.

FIG. 10 is a sectional view of an embodiment of the Centrifugal Fermentation Process when viewed parallel to the axis of rotation.

FIG. 11 are views of the rotor body of FIG. 10 when viewed parallel to the axis of rotation.

FIG. 12 is a cross-sectional view of one of the bioreactor chambers of FIG. 10.

FIG. 13 is a sectional view of the rotor body of FIG. 10 when viewed perpendicular to the axis of rotation.

FIG. 14 illustrates the axial channels and their termini in the rotating shaft of FIG. 10.

FIG. 15 is a sectional view of a representative high-performance end face seal.

FIG. 16 is a graphical and mathematical representation of the portion of the biocatalyst immobilization chamber of FIG. 12 which resembles a truncated cone.

FIG. 17 is a graph relating the flow rates and rotor speeds which provide for particle immobilization for the rotor body of FIG. 10 for particles of sedimentation rates of 0.001 and 0.01 nm/min at flow rates up to 10 mL/min.

FIG. 18 is a graph relating the flow rates and rotor speeds which provide for particle immobilization for the rotor body of FIG. 10 for particles of sedimentation rates of 0.1, 1.0, and 10.0 mm/min at flow rates up to 10 mL/min.

FIG. 19 is a graph relating the flow rates and rotor speeds which provide for particle immobilization for the rotor body of FIG. 10 for particles of sedimentation rates of 0.1, 1.0, and 10.0 mm/min at flow rates up to 100 mL/min.

FIG. 20 is a graph displaying the relationship between rotor size and volume capacity in a first embodiment of this invention.

FIG. 21 is a graph displaying the relationship between rotor size and volume capacity in a second embodiment of this invention.

FIG. 22 is a graph displaying the relationship between rotor size and rotational speed required to maintain a Relative Centrifugal Force of 100 g in embodiments of the process of this invention.

FIG. 23 is a block diagram of a centrifugal process configuration designed to allow serial processing of a precursor chemical through two centrifugal bioreactors.

FIG. 24 is an embodiment which may be employed for applications where the immobilized biocatalyst is in a complex consisting of a support particle to which the biocatalyst is attached.

FIGS. 25, 25A, 25B, and 25C illustrate a chamber system according to some embodiments of the present invention.

FIGS. 26A-D illustrate the shaft in side and cross-sectional views of the chamber system according to one embodiment of the invention.

FIG. 27 shows a front and side view of a reinforcement ring of the chamber system according to one embodiment of the invention.

FIGS. 28A-D are side and cross-sectional views of a sleeve of the chamber system according to one embodiment of the invention.

FIGS. 29A-B show side and end views of a flow diverter to be used with the chamber system according to one embodiment of the invention.

FIG. 30 is an end view of the shaft having a sleeve positioned thereon according to one embodiment of the invention.

FIG. 31 is an end view of the chamber system with the shield positioned for operation according to one embodiment of the invention.

FIGS. 32A-B show side and end views of a chamber of the chamber system according to one embodiment of the invention.

FIG. 33 is a side view of a chamber system according to an embodiment of the invention.

FIGS. 34A-B show end and side views of a chamber of the chamber system according to the embodiment of the invention shown in FIG. 33.

FIGS. 35A-B show end and cross-sectional views of one side of a chamber according to the embodiment of the invention shown in FIGS. 33-34.

FIGS. 36A-B show end and cross-sectional views of one side of a chamber according to the embodiment of the invention shown in FIGS. 33-34.

FIGS. 37A-B show cross-sectional views of one side of the chamber according to the embodiment of the invention shown in FIGS. 33-36.

FIGS. 38A-B show end and cross-sectional views of another side of a chamber according to the embodiment of the invention shown in FIGS. 33-34.

FIGS. 39A-B show side and end views of a portion of a shaft of the chamber system according to the embodiment of the invention shown in FIG. 33.

FIGS. 40A-B show side and end views of another portion of the shaft of the chamber system according to the embodiment of the invention shown in FIG. 33.

FIGS. 41A-E show side and cross-sectional views of a manifold sleeve of the chamber system according to the embodiment of the invention shown in FIG. 33.

FIG. 42 illustrates a perspective view of view of a chamber system according to an embodiment of the invention having a circular-shaped internal cavity.

FIGS. 43A-B illustrate a perspective view and a side view of a flow distributor according to an embodiment of the invention having circular-shaped discs.

FIG. 44 illustrates a perspective view of a circular-shaped disc of a flow distributor according to an embodiment of the present invention.

### DETAILED DESCRIPTION OF THE INVENTION

The development of the immobilization and culture process, described in U.S. Pat. Nos. 5,622,819; 5,821,116; 6,133,019; 6,214,617; and 6,660,509 to Herman, the entirety of each being herein incorporated by reference, has its origin in four distinct areas of knowledge. The function of the overall process depends on the use of information from all four areas for its proper function. These areas are: (1) Stoke's Law and the theory of counterflow centrifugation; (2) the geometrical relationships of flow velocity and centrifugal field strength; (3) Henry's Law of Gases; and, (4) the effect of hydraulic pressure on single and multicellular organisms and their cellular or subcellular components.

A main purpose of the process of this invention is immobilization of three-dimensional arrays of particles (cells, subcellular structures, or aggregated biocatalysts) and to provide them with a liquid environment containing dissolved gases which will maximize their viability and productivity. Such cells may include, but are not limited to, a prokaryotic cell, a bacterium, or a eukaryotic cell, such as algae cells, plant cells, yeast cells, fungal cells, insect cells, reptile cells and mammalian cells. The biocatalyst may be, but is not limited to, a subcellular component, an enzyme complex, and/or an enzyme complex immobilized on a solid support. The dissolved gases of the invention include but are not limited to air, O₃, NH₃, NO₂, Ar, He, N₃ and H₃ or any mixture thereof.

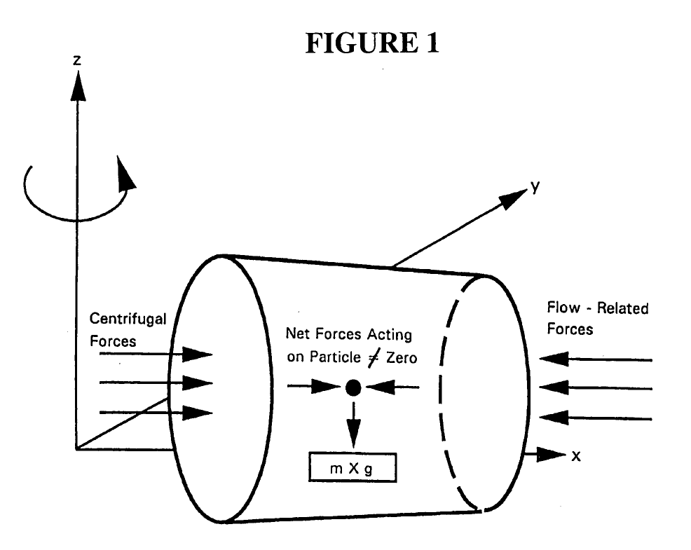

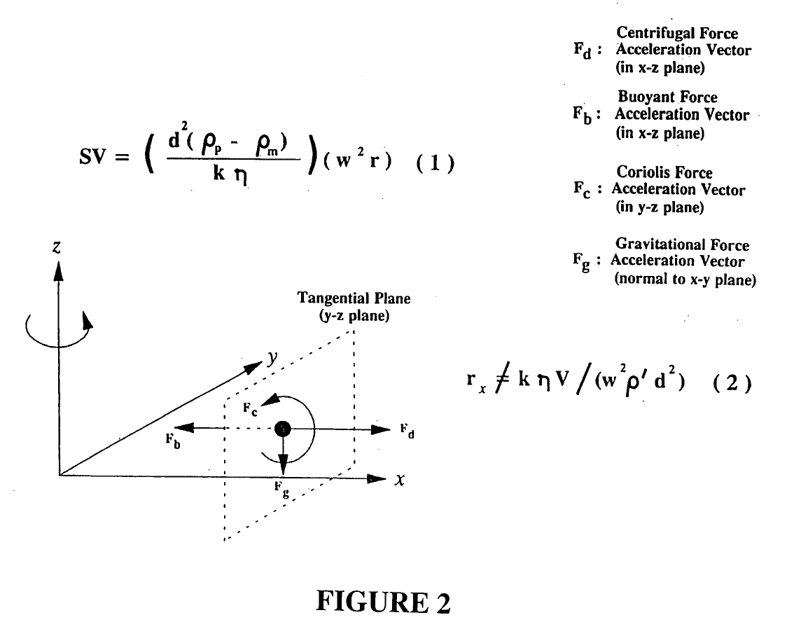

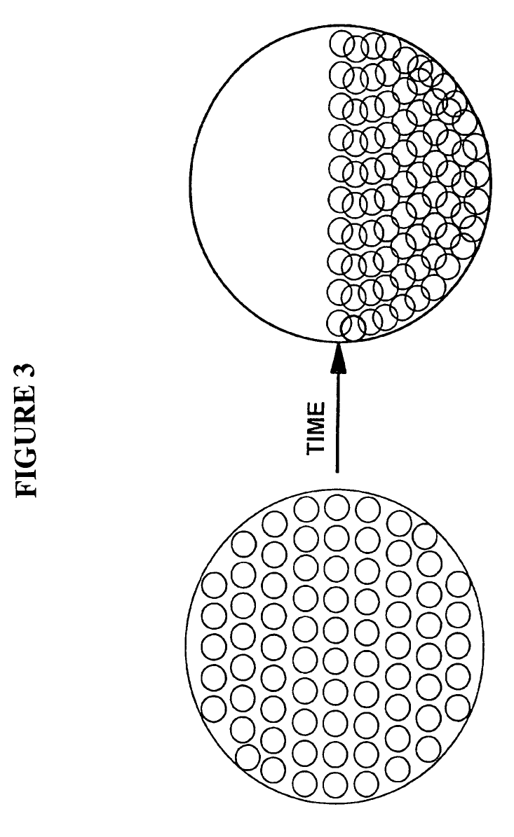

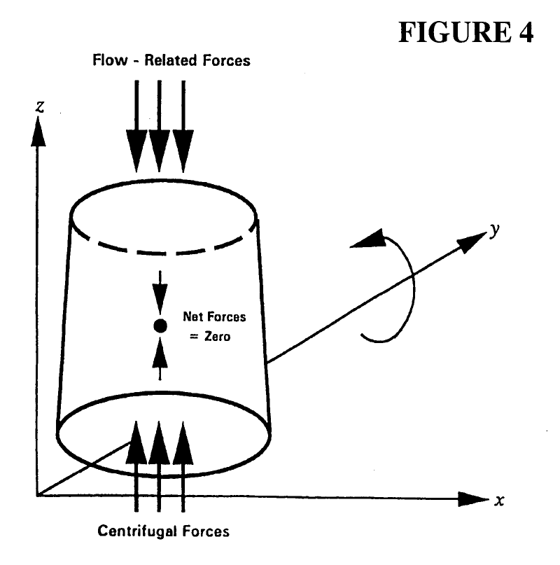

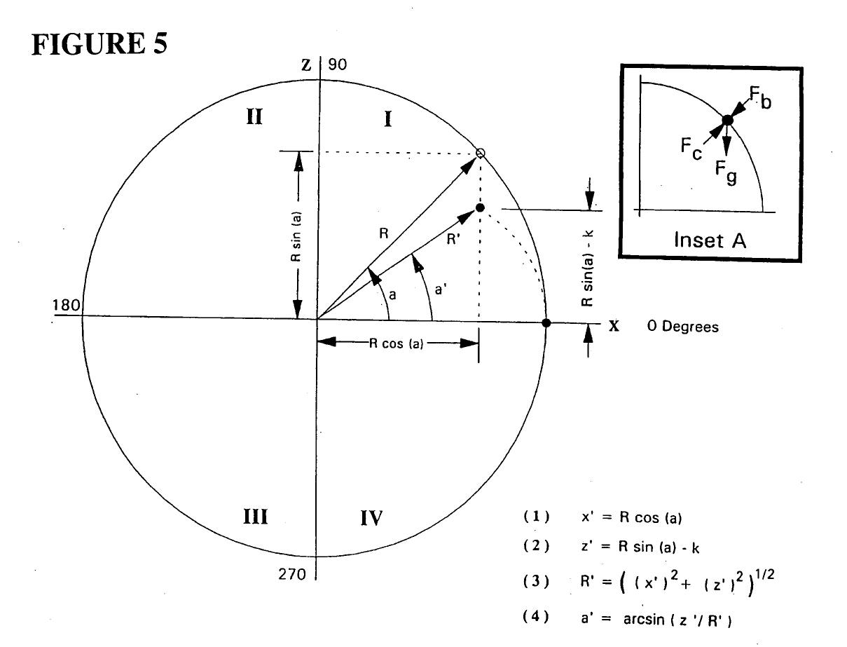

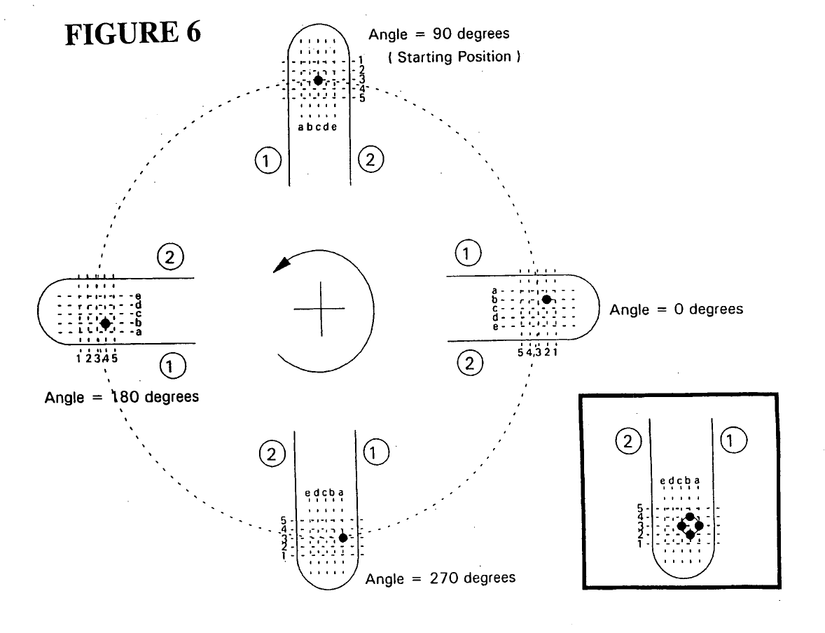

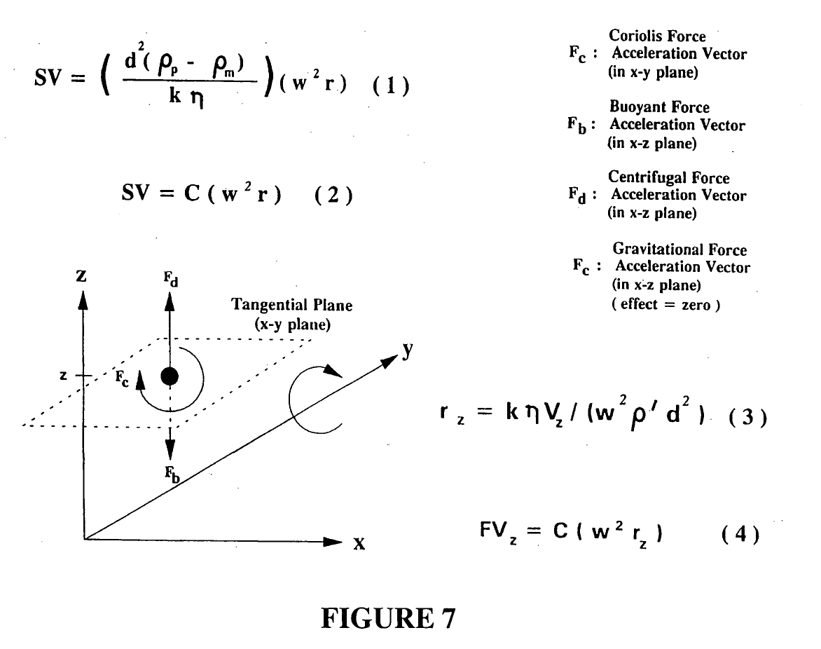

This process utilizes a novel modified form of “Counterflow Centrifugation” to immobilize particle arrays. A proper application of Stoke's Law in combination with provision for the effect of gravity which also acts on the immobilized particles results in a mathematical relationship which allows for the relative immobilization of high-density arrays of such particles. The effect of gravity discussed previously can be eliminated by an alternative choice of rotational axis. If rotation about the horizontal axis (y) is chosen instead of rotation about the vertical axis (z), as is most common in biological centrifugations, then the effect of gravity on immobilized particles will always be limited to action solely in the x-z plane. Since this is the same plane in which both the centrifugal as well as the liquid flow related forces are constrained to act, the motion of a restrained particle at any point in a rotational cycle is the resultant of the sum of the three types of forces acting upon it. FIGS. 1-7, described in U.S. Pat. No. 6,916,652 (Paragraphs 170-183) (which is incorporated by reference in this Application) more thoroughly detail and describe the theory underlying the various embodiments of the present invention.

As used herein, the terms “biocatalyst immobilization chamber”, “reactor chamber”, “bioreactor chamber”, “cell confinement chamber”, “centrifugal confinement chamber”, “centrifugal cell chamber”, “immobilization chamber”, “chamber”, “compartment”, or “confinement chamber” are all equivalent descriptive terms for the portion of the invention described herein where cells or biocatalysts are suspended by the described forces. Use of these equivalent terms does not imply an estoppel or limitation of the description of the invention.

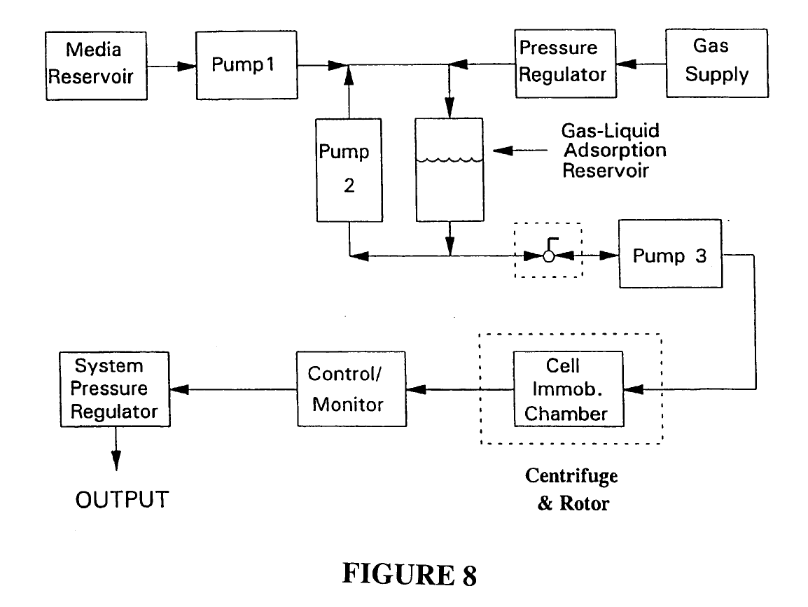

FIG. 8 is a block diagram which demonstrates one method by which the maintenance of such a system having gas dissolved in a liquid at hydraulic pressures greater than ambient may be effected. In this system, the indicated pumps are all positive displacement pumps. That is, liquid is constrained to motion through the pumps in the directions indicated by the arrows. Pump 3 is the primary feed pump which moves liquids into and out of the cell immobilization chamber which is located in a centrifuge rotor. The raising of the hydraulic pressure in the circuit containing Pump 3 and the cell immobilization chamber is accomplished by placing a liquid pressure regulator, the system pressure regulator, at a position in the circuit downstream of the cell immobilization chamber. Thus, the setting of a pressure limit higher than ambient on the system pressure regulator results in no liquid flow through this circuit until the positive displacement pump, Pump 3, moves enough liquid into the circuit to raise the system hydraulic pressure to a value near this setting. Once an equilibrium system pressure is established, the pressurized liquid downstream of Pump 3 will flow continuously at a rate set by control of Pump 3.

To dissolve an appropriate amount of a desired gas into the liquid input to Pump 3, a Gas-Liquid Adsorption Reservoir is placed in the input line leading to Pump 3. Non-gassed liquids are moved from the Media Reservoir into the Gas-Liquid Adsorption Reservoir by means of Pump 1. Quantities of the desired gas (air or oxygen, for example) are, at the same time, let into the Gas-Liquid Adsorption Reservoir through a pressure regulator set for the gas pressure required to insure the solubilization of the desired concentration of the dissolved gas into the liquid. Note that, in the steady-state, it is necessary that Pump 1 be operated at the same flow rate set for Pump 3. Pump 2 is a recirculation pump which is operated at a flow rate higher than that of Pumps 1 and 3. Pump 2 is used to increase the contact between the gas and liquid phases of the Gas-Liquid Adsorption Reservoir so that a desired concentration of gas dissolved in the nutrient liquid is maintained in the bulk of the volume of liquid in the Gas-Liquid Adsorption Reservoir. It is essential, because of the nature of positive displacement pumps, that the magnitude of the system pressure set with the System Pressure Regulator be higher than the pressure magnitude set in the Gas-Liquid Adsorption Reservoir. To make available, at any time, a sufficient volume of liquid equilibrated with the desired concentration of gas(es), a valve on the input to Pump 3 may be utilized to allow such equilibration to occur prior to any actual use. Similarly, by means of switching valves, the liquid input to Pump 3 may be changed from that indicated in FIG. 8 to any other input reservoir desired, subject to the constraint that the hydraulic pressure of such a reservoir be lower than the value of hydraulic pressure set by the System Pressure Regulator.

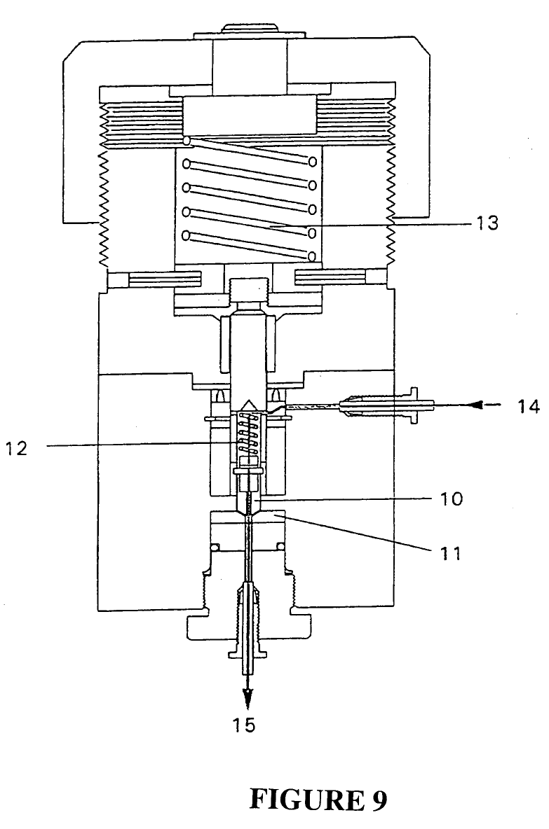

FIG. 9 is a depiction of a representative, commercially-available liquid pressure regulator. A flow of liquid 14 into the pressure regulator is obstructed by a spring-loaded needle valve 10 which presses against a seat 11. When the hydraulic pressure of the input liquid becomes great enough, the needle valve 10 is displaced from the seat 11 and a flow can then exit (as indicated by line 15) the pressure regulator. The fixed pressure exerted by the needle valve spring 12 can be adjusted by increasing or decreasing the pressure exerted by the adjustable spring 13.

It should be obvious that the block diagram of FIG. 8 is a representation of one of many process flow configurations which may be employed to flow a gas-free pressurized liquid through a centrifugal bioreactor chamber. In particular, one may envision many different methods of insuring adequate mixing of gas and liquid to effect the solubilization of a measured quantity of gas into the liquid. What is central to the process of this invention is: (1) that the liquid circuit comprising the bioreactor chamber and the liquid transport lines (into and out of the bioreactor chamber) be operated at a hydraulic pressure greater than ambient pressure; (2) that there be provision for the solubilization of a desired quantity of a gas into the liquid prior to its insertion into the liquid circuit leading to the bioreactor chamber(s); and (3) that the system hydraulic pressure be maintained at a high enough value to keep both the input gas(es), as well as the respiratory gas(es) which may be produced by biological systems in solution throughout the liquid circuit, upstream of the system pressure regulator and downstream of Pump 3. Hydraulic pressures of 100-2000 psig have proved sufficient to maintain a liquid environment for all possible conditions of cell density and cell number.

There will be no measurable deleterious effects on the culture of animal cells or micro-organisms or their subcellular constituents as a result of the necessity to increase the hydraulic pressure of their environment in the biocatalyst immobilization chamber at hydraulic pressures above 1 ATM and below 10,000 psig. The successful culture of living cells using bioreactor headspace pressurization is a proven and established culture method, albeit limited in scope to pressures of less than 50 psig (see Yang, J. and Wang, N. S. (1992) Biotechnol. Prog. 8, 244-251 and references therein). At hydraulic pressures of 15,000 to 30,000 psig some disassociation of noncovalent protein complexes has been observed, although pressures of more than 90,000 psig are required to denature monomeric proteins (Yarmush, et al. (1992) Biotechnol. Prog. 8, 168-178). It is a seldom appreciated, but well known fact that living cells (and their constituent parts) are unaffected by, and indeed cannot sense hydraulic pressure magnitudes below those limits outlined above. This may best be appreciated in considering the effects of hydraulic pressure on marine organisms. For every 10 meters of depth under the sea, approximately one atmosphere (14.7 psig) of overpressure is gained. Thus, for example, benthic organisms exhibiting biochemical processes and metabolic pathways identical to their shallow-water and terrestrial counterparts inhabit ecological niches and proliferate at hydraulic pressures of more than 3000 pounds per square inch. Similarly, the hydraulic pressure under which terrestrial mammalian cells exist is greater than ambient, ranging from about 90 to 120 mmHg greater than ambient in man, for example. The explanation for the “invisibility” of hydraulic pressure in biological systems can be understood if it is realized that hydraulic pressure in aqueous systems has, as its “force carrier,” the water molecule. Since the lipid bilayer which forms the boundary membrane of living cells is completely permeable to water molecules, an applied hydraulic pressure in aqueous systems is transmitted across the boundary membranes of cells or subcellular organelles by the movement of water molecules with the result that the interior(s) of cells rapidly equilibrate to an externally-applied aqueous hydraulic pressure.

There are situations in which hydraulic pressures are deleterious to living cells. For example, if a pressure field in an aqueous system is varied at high frequency, then it is possible to cause cell disruption by means of pressure differentials across the cell boundary membrane. However, the frequency required for such lethal effects is quite high; on the order of thousands of cycles per second. As long as the pulsatile pressure of pumping in the process of this invention is kept below such a limit there is no effect on cell viability for even the most fragile of cells as a result of pressure fluctuations. In addition, cell replication is completely unaffected by culture at increased hydraulic pressure.

The problem of the introduction and withdrawal of pressurized liquid flows into and out of a rotating system has been solved by innovations in seal design over the past twenty years. High performance mechanical end-face seals are available which are capable of operation at rotational rates in excess of 5000 revolutions per minute while maintaining a product stream hydraulic pressure of more than 2000 psig. Such seals are available from Durametallic Corporation (2104 Factory Street, Kalamazoo, Mich. 49001). Such high-performance mechanical seals have leakage rates below 5 liters per year, can be cooled by pressurized refrigerated liquids of which inadvertent leakage into the product stream at the above leakage rates will have no effect on biological systems, and can be operated in a manner which provides for the maintenance of absolute sterility in the product stream. The use of mechanical end-face seals in centrifugal bioreactor systems (see U.S. Pat. Nos. 4,939,087 and 5,151,368) results in a perceived necessity for the connection of flexible tubing (and complicated mechanisms for its “untwisting”) in conventional designs. Such designs are, as a result, limited to: (1) hydraulic pressures near one atmosphere as a consequence of tube flexibility requirements; and (2) low rotational speeds and short bioreactor run times as a result of the vigorous motion of these flexing connections. The use of moderm high performance mechanical end-face seals eliminates drawbacks to centrifugal bioreactor performance.

Immobilization of three-dimensional arrays of particles in a force field, which is comprised of outwardly-directed centrifugal forces which are opposed by inwardly-directed liquid flow forces has been described. The effect of gravitational forces which act, inevitably, on even the smallest and lightest of particles over prolonged time periods can be essentially negated and reduced to a small periodic “vibration in place” by the proper choice of rotational axis. The disruptive effects of the possible introduction of gases into this system have been accounted for by raising the hydraulic pressure of the liquid system to values which assure that such otherwise gaseous chemicals will remain dissolved in the flowing liquid. It has been emphasized that the necessary increase in hydraulic pressure will have no effect on biological units such as cells, microorganisms, or their subcellular constituents.

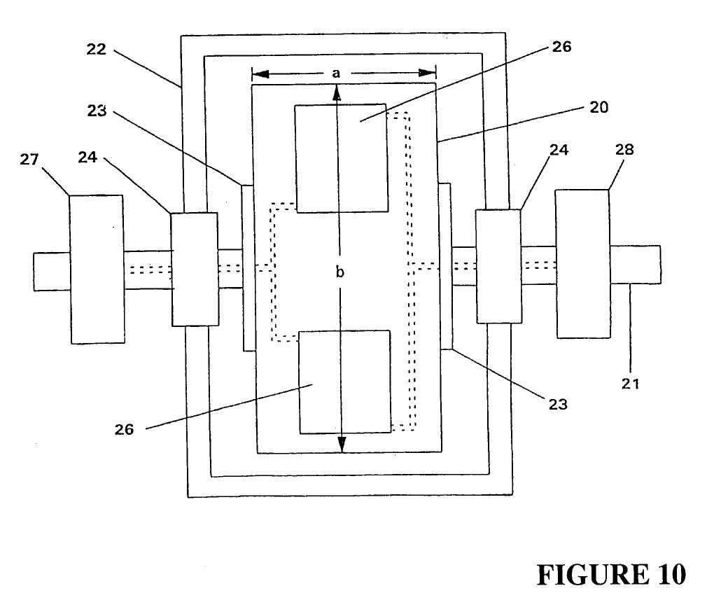

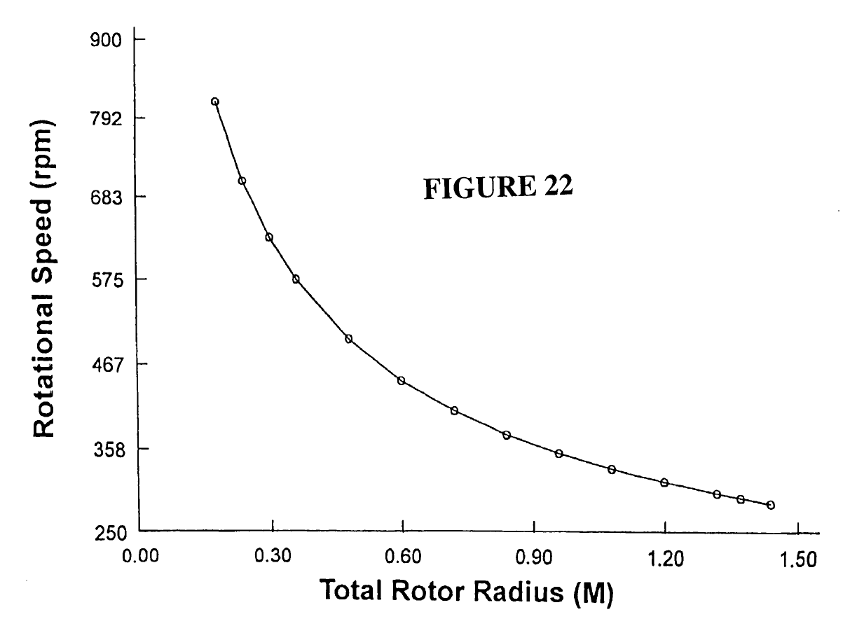

FIG. 10 depicts the components of a first embodiment of the present invention. A cylindrical rotor body 20 is mounted on a horizontal, motor-driven rotating shaft 21 inside a safety containment chamber 22 bounded by metal walls. The rotor body 20 is fixed in position on the rotating shaft 21 by means of locking collars 23. The rotating shaft 21 is supported on either side of the rotor body 20 by bearings 24. The rotating shaft 21 extends outside the safety containment chamber 22 for a distance and ends in a terminal bearing and end cap 29 mounted in an external housing 25. Liquid flows are introduced into and removed from bioreactor chambers 26 mounted in the rotor body 20 by means of a liquid input mechanical end-face seal 28 and a liquid output mechanical end-face seal 27 which communicate with liquid channels (50, 51 in FIG. 22) within the rotating shaft 21. Typical dimensions for an example rotor body 20 (a=36 cm and b=15 cm) are entirely reasonable and comparable to rotor dimensions known to those skilled in the art.

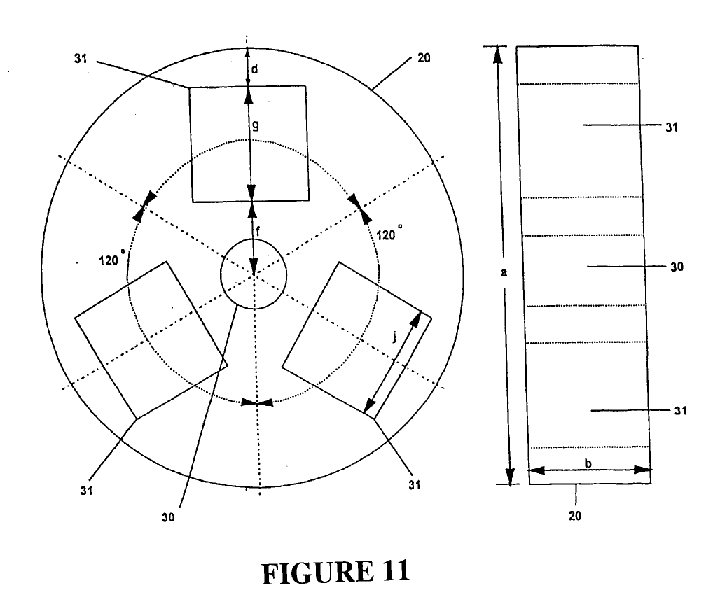

FIG. 11 shows two views of the rotor body 20 of FIG. 10. The rotor body 20 is machined with a shaft mounting channel 30 through its center to allow its mounting on the rotating shaft (21 in FIG. 10) and is machined to have mounting recesses 31 into which three rectangularly-faced demountable bioreactor chambers may be placed.

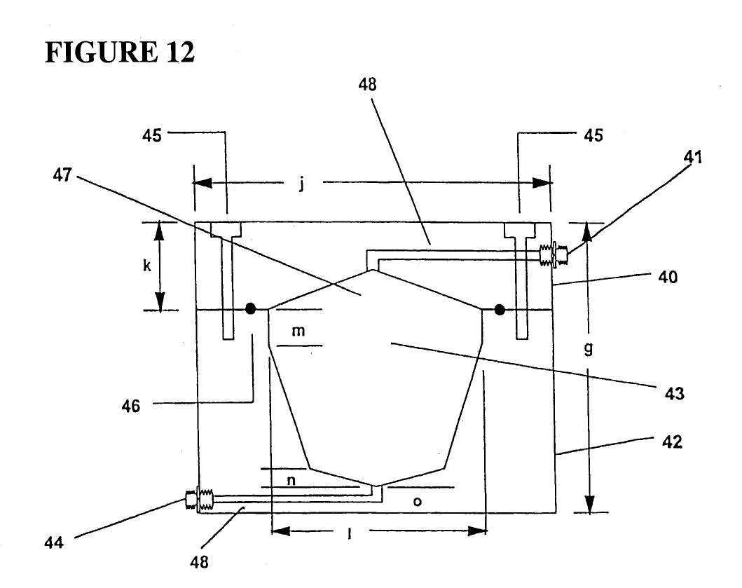

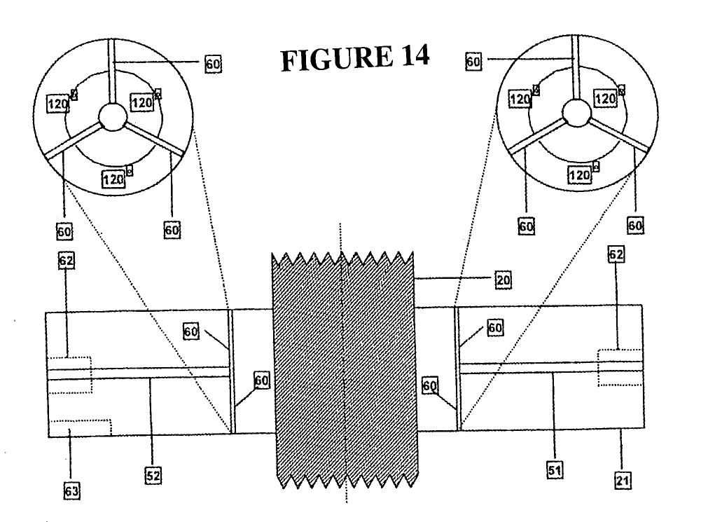

FIG. 12 is a depiction of one of the bioreactor chambers of FIG. 14 (26 in FIG. 10). The bioreactor chamber (26 in FIG. 10) is rectilinear in section and is composed of a top piece 40 and a bottom piece 42 of thick-walled metal. The top piece 40 contains a machined conical recess 47 and a machined passage 48 terminating in an output compression fitting 41 by which liquid may be removed from the bioreactor chamber (26 in FIG. 10). The bottom piece 42 is made from the same metal as the top piece 40 and has been internally machined to form a biocatalyst immobilization chamber 43 of a desired geometric shape. The shape of the biocatalyst immobilization chamber 43 is that of a truncated cone with a short cylindrical volume at its top face and a short conical volume at its bottom face. A machined passage 48 terminating in an input compression fitting 44 allows liquid input into the biocatalyst immobilization chamber 43. The top piece 40 and the bottom piece 42 of the biocatalyst immobilization chamber 43 are bolted together by means of countersunk assembly screws 45 and sealed against an internal positive hydraulic pressure by means of one or more o-ring compression seals 46. In the case of certain animal cell cultures where contact between the immobilized cells and the interior metal walls of the biocatalyst immobilization chamber 43 should be avoided, it may be expedient to provide suitable conical inserts of, for example, polyethylene, to prevent such contact. Alternatively, the interior of the biocatalyst immobilization chamber 43 might be coated with an appropriate lining material to provide the same effect.

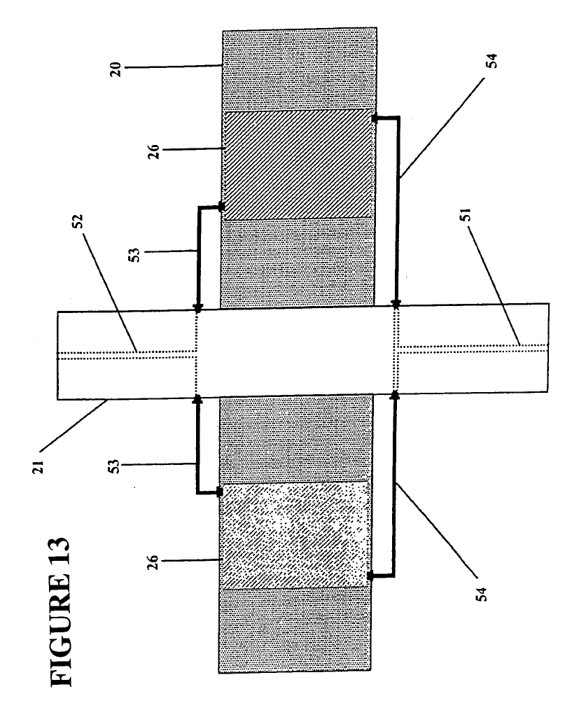

FIG. 13 is a transverse sectional view through the rotor body 20 of FIG. 10 and the rotating shaft 21 of FIG. 10 parallel to the axis of rotation. The output liquid transport lines 53 are metal tubes which communicate with the bioreactor chambers 26 and the centrally-located axial liquid output channel 51 through output compression fittings (41 in FIG. 12). The input liquid transport lines 54 are metal tubes which communicate with the bioreactor chambers 26 and the centrally-located axial liquid input channel 52 through input compression fittings (44 in FIG. 12).

FIG. 14 is a view of the rotating shaft 21 of FIG. 10 on which the rotor body 20, the liquid output mechanical end-face seal (27 in FIG. 10), and the liquid input mechanical end-face seal (28 in FIG. 10) are mounted. The rotating shaft 21 contains a centrally-located axial liquid output channel 51 and a centrally-located axial liquid input channel 52. The centrally-located axial liquid output channel 51 (typically 1/8 ″ diameter) transports the liquid output of the bioreactor chambers (26 in FIG. 10) to the liquid output mechanical end-face seal (27 in FIG. 10) by means of three short radially-directed passages 60 while the centrally-located axial liquid input channel 52 (also 1/8 ″ diameter) conveys liquid from the liquid input mechanical end-face seal (28 in FIG. 10) into the bioreactor chambers (26 in FIG. 10), also by means of three short radially-directed passages 61. The centrally-located axial liquid output channel 51 and the centrally-located axial liquid input channel 52 extend from each end of the rotating shaft 21 to the region where the rotor body 20 is located. Each end of the rotating shaft 21 has a threaded recess 62 which is formed to accept threaded liquid mechanical seals. The leftmost end of the rotating shaft 21 is also machined to provide a keyway 63 to which a motor drive pulley (not shown) may be attached.

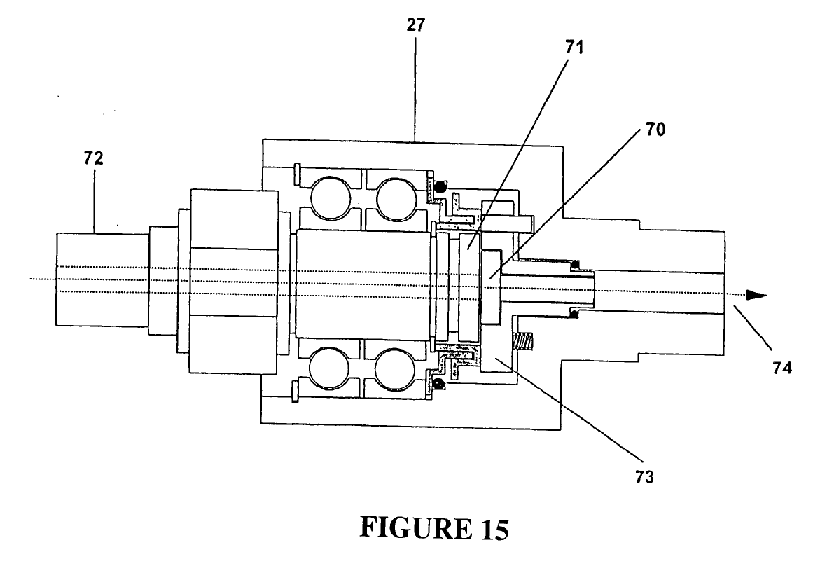

FIG. 15 is a view of a typical liquid output mechanical end-face seal assembly such as the liquid output mechanical end-face seal 27, shown in FIG. 10. The rotating part 72 of the liquid output mechanical end-face seal 27 is threaded into the threaded recess (62 in FIG. 14) in the leftmost end of the rotating shaft (21 in FIG. 14). A seal between the rotating and stationary portions of the liquid output mechanical end-face seal 27 is provided by the contact of the stationary seal face 70 against the rotating seal face 71. In the case of high performance mechanical end-face seals utilizable in the process of this invention, where consideration must be made for the resultant centrifugal forces which act on the seal components, all spring elements are located in the stationary portion of the seal assembly. While the seal assembly shown in FIG. 15 is a single seal, double and/or tandem end-face seal configurations may prove more advantageous in prolonged usage. When such a seal assembly is mounted on the rotating shaft (21 in FIG. 13) of the invention, aqueous liquids may be pumped out of the stationary part 73 of the liquid output mechanical end-face seal assembly via compression fittings and the pumped liquid will follow the path indicated by the dotted line 74 to make communication with the centrally-located axial liquid output channel (51 in FIG. 13) which transports the liquid away from the bioreactor chambers (26 in FIG. 13) mounted in the rotor body (20 in FIG. 13).

To obtain data for an analysis of the performance of a rotor body (20 in FIG. 10) of the dimensions and configuration outlined in FIGS. 10-11 and 13-14 and containing demountable rectilinear biocatalyst immobilization chambers 43 like those depicted in FIG. 12, it was necessary that several scale dimensions and boundary equations be chosen arbitrarily and used to determine the operating characteristics of the second embodiment of the invention. The immobilization boundary equations chosen are those listed in Equations 1 and 2 of FIG. 15 of U.S. Pat. No. 6,660,509.

In the embodiments of the present invention, described above, a portion of the geometry of the biocatalyst immobilization chamber (43 in FIG. 12) is that of a truncated cone. As is shown in FIG. 10, the dimensional problem of determining the “aspect ratio” (the ratio of the small radius of the truncated cone 110 to the large radius of the truncated cone) of the biocatalyst immobilization chamber (43 in FIG. 12) due to boundary condition constraints can be reduced to an examination of the geometrical relationships between the large and small radii of the truncated cone 110 and the height of the truncated cone 110.

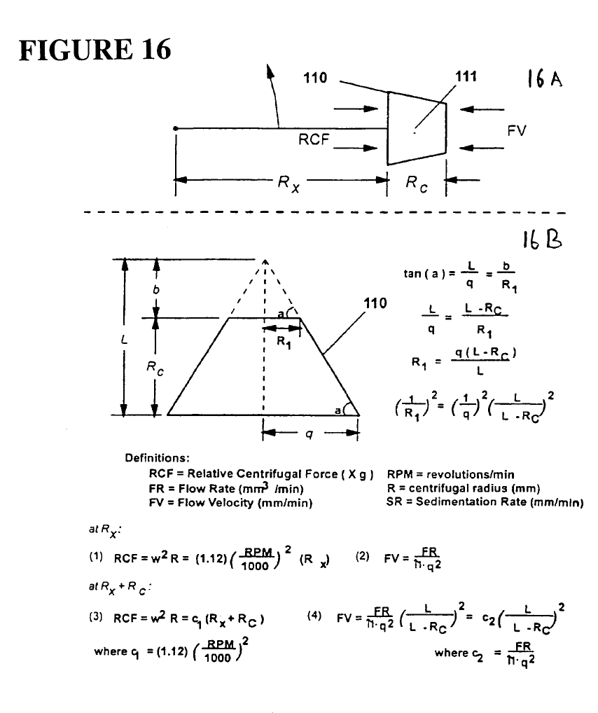

FIG. 16A is a sectional view, through the plane of rotation, of the portion of the biocatalyst immobilization chamber (43 in FIG. 12) which resembles a truncated cone 110. The truncated cone 110 has a proximal face which is located a distance of Rₓ from the center of rotation. The truncated length of the cone is R_{c} A Relative Centrifugal Force (RCF) acts to cause translation of a particle 111 in the biocatalyst immobilization chamber (43 in FIG. 12) to longer radii, while liquid flow forces (FV) act to cause translation to shorter radii. Equation (1) of FIG. 16B is an expression for the magnitude of the Relative Centrifugal Force (RCF) at radial length (Rₓ) in terms of the Rotor Speed (RS). Equation (2) is an expression for the magnitude of the Flow Velocity (FV) at radial length (Rₓ) in terms of the liquid Flow Rate (FR) and the large radius (q) of the truncated cone 110. Equation (3) is an expression for the magnitude of the Relative Centrifugal Force (RCF) at radial length (Rₓ+R_{c}) in terms of the Rotor Speed (RS). Equation (4) is an expression for the magnitude of the Flow Velocity (FV), at radial length (Rₓ+R_{c}), in terms of the liquid Flow Rate (FR) and the given dimensions of the truncated cone 110 and its sections. To determine the “aspect ratio” of the truncated cone 110 which will satisfy certain boundary conditions, given the physical dimensions of the rotor body (20 in FIG. 10), we have chosen to express the radius of the small end (R1) of the truncated cone 110 in terms of the length (L) of a non-truncated version of the truncated cone 110. This non-truncated version is shown in dotted lines in FIG. 16B.

The desired boundary conditions are: (1) that the product of the intrinsic Sedimentation Rate (SR) of the immobilized particle due to gravity and the applied centrifugal field (RCF) be exactly equal to the magnitude of the liquid flow forces (FV) at the most distal portion of the biocatalyst immobilization chamber (43 in FIG. 12); and (2) that this product be twice the magnitude of the liquid flow forces (FV) at the most proximal portion of the biocatalyst immobilization chamber (43 in FIG. 13). Thus: at centrifugal radius=Rₓ+R_{c}; (SR)×(RCF)=FV at centrifugal radius=Rₓ; and (SR)×(RCF)=2×FV. Substituting into these equations the dimensional specifications for RCF and FV, we now have two simultaneous equations which relate the liquid Flow Rate (FR), the Rotor Speed (RS), and the dimensions of the biocatalyst immobilization chamber (43 in FIG. 12): (SR)C₁(Rₓ+R_{c})=C₂(L/(L−R_{c})) (Eqn. 1) and (SR)C₁(Rₓ)=2×C₂ (Eqn. 2).

To arrive at a solution to these equations, we will make the following substitutions which are based on the physical dimensional limits of the example rotor system: Rₓ=90 mm; R_{c}=30 mm; q=30 mm. The simultaneous equations now become: (SR)C₁(120)=C₂(L/(L−30))² (Eqn. 1) and (SR)C₁(Rₓ)=2×C₂ (Eqn. 2). Substituting Eqn. (2) into Eqn. (1) yields: (L/(L−30))²=240/90. Solution of this quadratic expression yields L=77.4 mm and: (L/(L−30))²=2.67. Since it was earlier determined that: R₁=(q(L−R_{c}))/L, the smaller radius of the truncated cone which satisfies the boundary conditions is: R₁=18.4 mm. Now, the two simultaneous equations become: (SR)C₁(120)=C₂(2.67) (Eqn. 1) and (SR)C₁(90)=C₂(2) (Eqn. 2); and, by subtracting (2) from (1) and collecting terms, we arrive at: (SR)(30)C₁=(0.67)C₂. Substitution into this equation the values of C₁ and C₂ yields: (SR)(30)(1.12)(RPM/1000)²=(0.67)((FR)/(π×q²)). Now we have an expression which satisfies the desired boundary conditions and physical dimensional constraints in terms of the controllable variables, RS and FR: (√SR)(RPM)=(2.65)(√FR).

Thus, once the physical dimensions of the rotor system as well as those of the biocatalyst immobilization chamber have been determined, the range of Rotor Speeds (RS) and the system liquid Flow Rates (FR) which will constrain the particles to immobility in the bioreactor will follow a simple relationship which is dependent only on the intrinsic Sedimentation Rate (SR) of the object particle due to gravity. Note that, under the above conditions, the maximal volume of immobilization is approximately 56 mL per bioreactor chamber.

This method and apparatus for containing a biocatalyst comprises the step of containing the biocatalyst in a bioreactor chamber placed in a centrifugal force field where the centrifugal force field is oriented in a plane parallel to the plane in which the force of gravity acts. The centrifugal force field is diametrically opposed by a continuously flowing liquid at hydraulic pressures greater than the ambient barometric pressure.

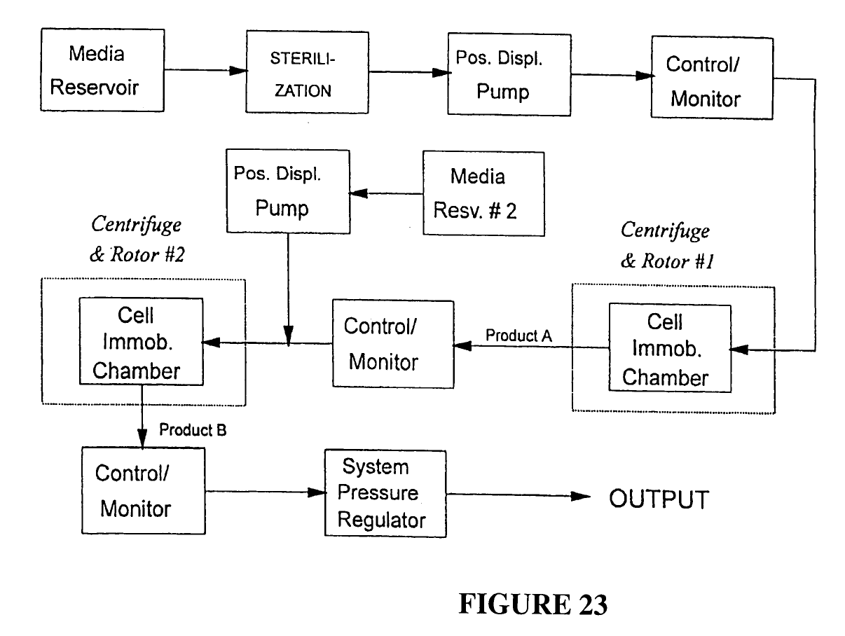

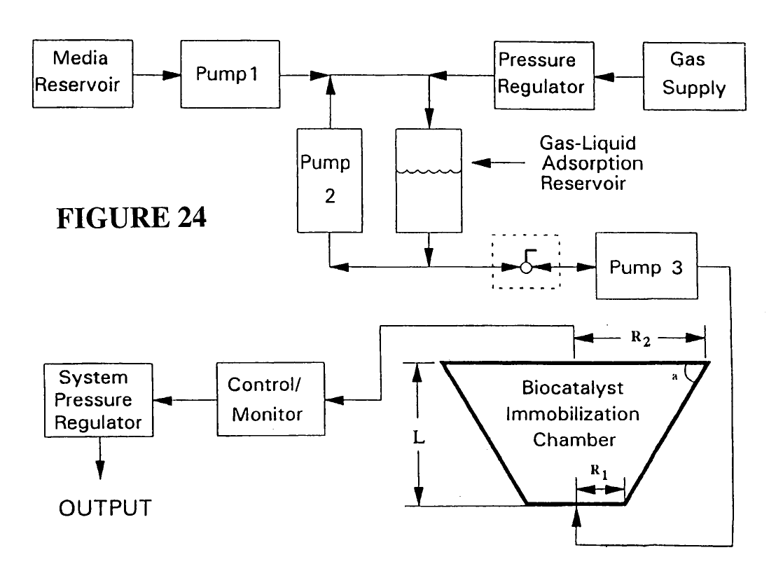

FIGS. 23 and 24 depict components of other embodiments of the present invention. This embodiment is a design which may be employed for applications where the immobilized biocatalyst is in a complex consisting of a dense inert support particle to which the actual biocatalyst is attached. In such an application, the buoyant force acting on the biocatalyst/support complex as a result of nutrient liquid flow can be negated, and thus immobilizing the biocatalyst/support complex, by the vertical alignment of the biocatalyst immobilization chamber so that the earth's gravitational field acts on the biocatalyst/support complex to provide the required counter-acting force. Further, the range of flow rates which can be accommodated in this system is in no way limited since the buoyant force which must be countered is the nutrient liquid flow velocity. The magnitude of the flow velocity can be varied through a desired range by varying the cross-sectional diameters and the aspect ratio of those diameters as necessary. The relative centrifugal field in this case is close to 1×g (that provided by the earth's gravitational field). Thus, the required applied centrifugal field, in this case, is zero.

In the embodiment shown in FIG. 24, nutrient liquids, which have been pressurized and have dissolved in this liquid the appropriate quantities of a nutrient which is gaseous at ambient pressure, are pumped into a stationary biocatalyst immobilization chamber fed by the main feed pump, Pump 3. The continuation of the liquid flow as it exits the biocatalyst immobilization chamber is fed through control and monitoring sensors and through a system pressure regulator which maintains the elevated hydraulic pressure of the system. The ratio of R₁ to R₂ is dependent on the desired flow velocity boundary conditions and can vary downward from 1.0 to any desired fraction thereof. R₁ is not limited in dimension: its magnitude is determined by the size of the liquid flow rate which is desired. L, the height of the immobilization chamber, is not limited in dimension: its magnitude is determined by the desired retention time of a nutrient liquid bolus as it passes through the biocatalyst immobilization chamber.

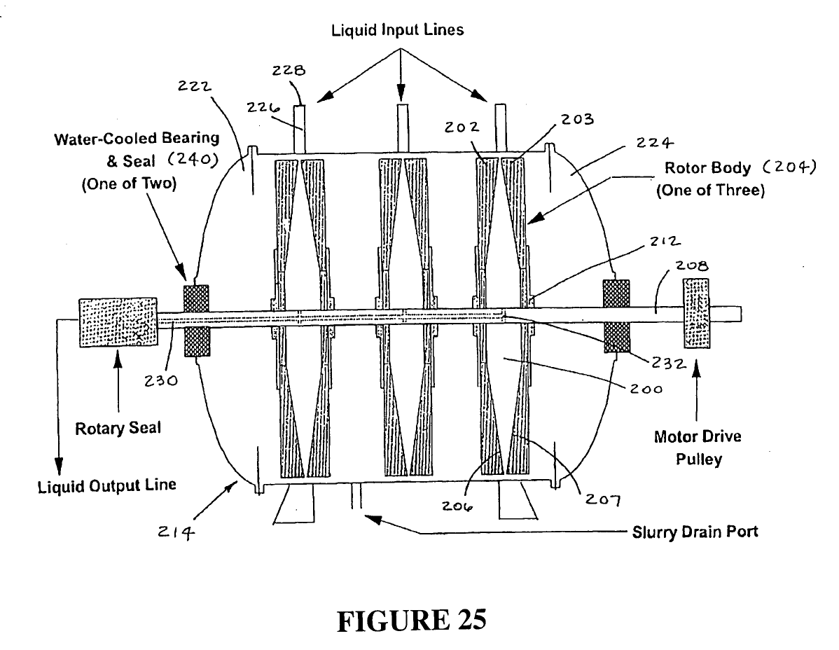

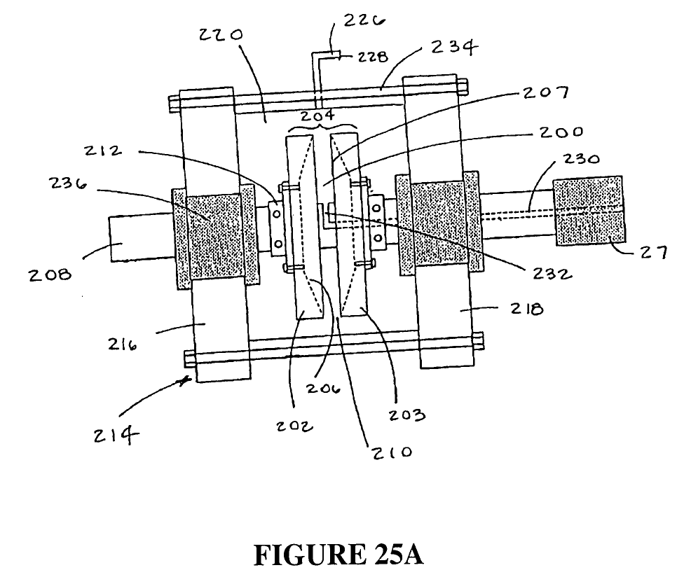

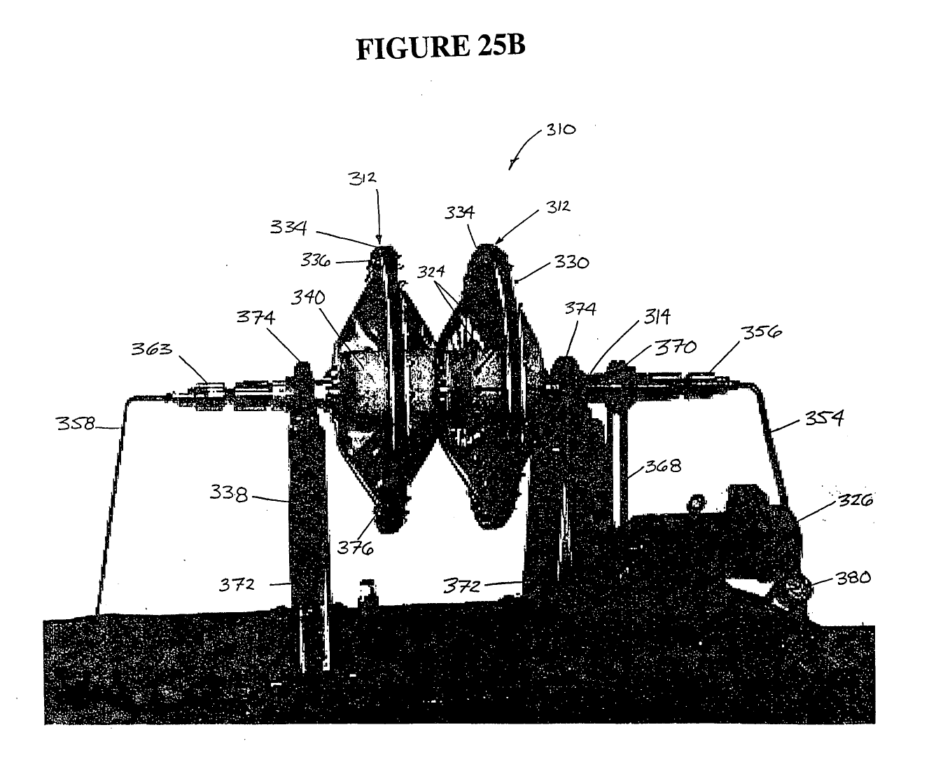

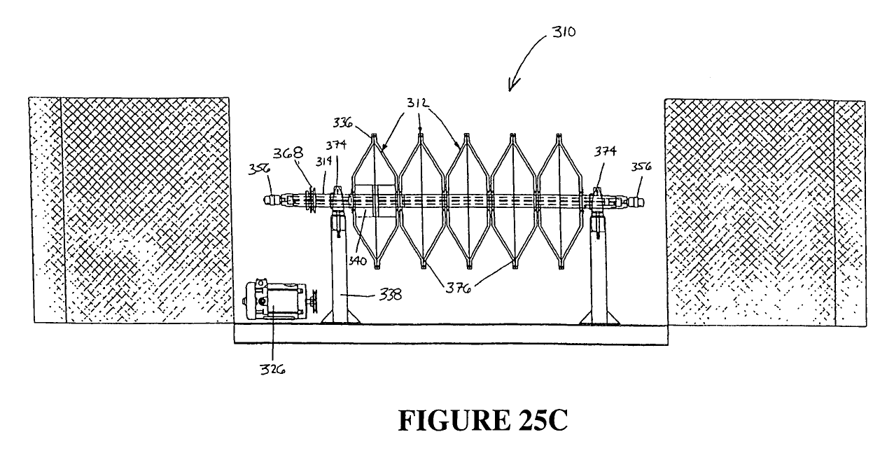

To obtain data for an analysis of the performance of a biocatalyst immobilization chamber of the embodiment in FIG. 24, (dimensions denoted by letters), it was necessary that several scale dimensions and boundary equations be chosen arbitrarily and used to determine the operating characteristics of an embodiment of the invention. The immobilization boundary equations (both the top and bottom boundary equation) chosen is that listed in Equation 1 of FIG. 15 of U.S. Pat. No. 6,660,509. The biocatalyst immobilization chamber dimensions chosen for this example and indicated by letter in FIG. 25 are as follows: R₁:5.0 cm; R₂:5.1 cm; L:61.0 cm.

A biocatalyst immobilization chamber of the above dimensions was loaded with 100 mL of 30-50 mesh peanut shell charcoal (density: 3.5 gm/mL). The system configuration of FIG. 24 was established and, at a liquid flow rate of 120 mL/min, an equilibrium between the flow velocity-derived buoyant forces and the intrinsic sedimentation rate of the individual charcoal particles at 1×g relative gravitational field resulted in a stable, immobilized, three-dimensional array. Note that small flow rate variations near the nominal value chosen resulted in small increases or decreases in the immobilized array density and volume, while large changes in flow rate require that R₁, R₂, and L be changed, thus requiring separate biocatalyst immobilization chamber sizes to accommodate different flow rate regimes. While the charcoal particles were found suitable for the attachment of a number of bacterial genera, the type of inert particle employed for a specific biocatalyst immobilization purpose are limited only in the compatibility with the biocatalyst and the liquid environment of the system.

There are many alternative shapes for the biocatalyst immobilization chambers which are contemplated in this invention. One such alternative embodiment is a biocatalyst immobilization chamber having its inner space in the shape of a right circular cone with a major axis which is aligned parallel to the applied centrifugal force field and which has a large diameter which is nearer to the axis of rotation than is its apex.

Another alternative embodiment is a biocatalyst immobilization chamber having its inner space in the shape of a right circular cone which has a major axis which forms an angle of between 0 and 90 degrees with the applied centrifugal force field. Also included in the invention is a biocatalyst immobilization chamber having its inner space in the shape of a truncated right circular cone which has a major axis which is aligned parallel to the applied centrifugal force field and which has a large diameter which is nearer to the axis of rotation than is its minor diameter.

FIG. 25 depicts an alternative embodiment of a rotor body of the invention. Instead of forming individual chamber positioning recesses in a rotor body to hold the bioreactor chambers, each bioreactor chamber 200 is formed by a pair of rotor disks 202, 203 that form a rotor body 204. The rotor disks 202, 203 may be made from any material sufficiently strong to withstand the degree of centrifugal force contemplated by the invention, such as aluminum, stainless steel, or plastic.

At least one face 206, 207 of each disk 202, 203 is contoured so that adjacently positioning the disks 202, 203 so that the contoured faces 206, 207 oppose each other forms a chamber 200 between the disks 202, 203. The desired geometrical shape of the chamber 200 is first calculated using the methods disclosed herein. Once the desired shape is known, a face 206, 207 of each disk may be contoured and the disks 202, 203 positioned to form the desired chamber 200 between the disks 202, 203.

The disks 202, 203 are then mounted on a preferably semi-hollow rotating shaft 208. The rotor disks 202, 203 of each rotor body 204 preferably do not touch so that a gap 210 is formed between the disks 202, 203. The disks 202, 203 may be fixed in position on the rotating shaft 208 by any appropriate fixing means, such as, for example, locking collars 212. The fixing means ensure that the disks 202, 203 of the rotor body 204 remain separated by the desired distance during rotation of the shaft 208. The fixing means preferably also allow adjustment of the gap 210 between the rotor disks 202, 203. As increased volume or production capacity is needed, the diameter of the rotor disks 202, 203 and the gap 210 between the rotor disks 202, 203 may be increased.

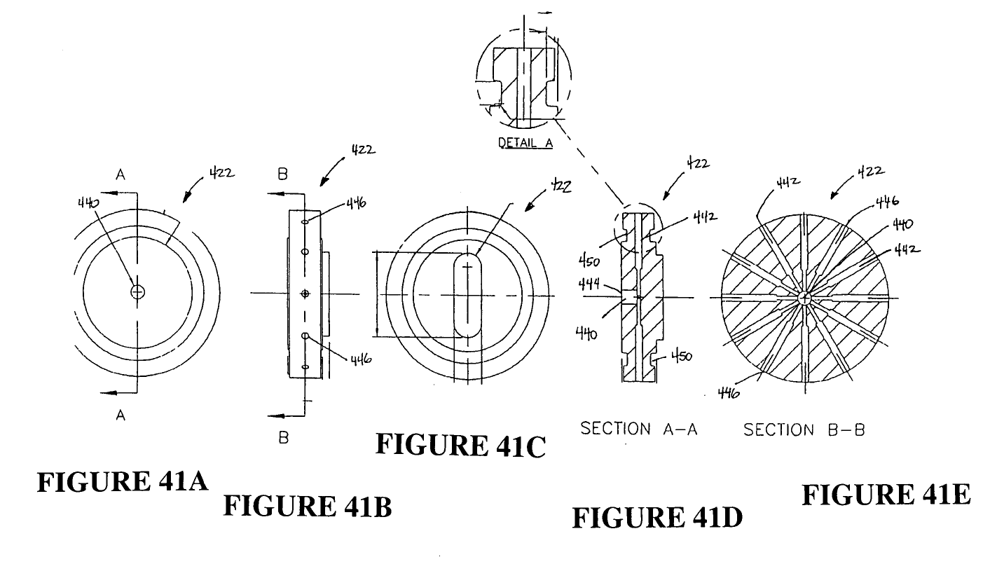

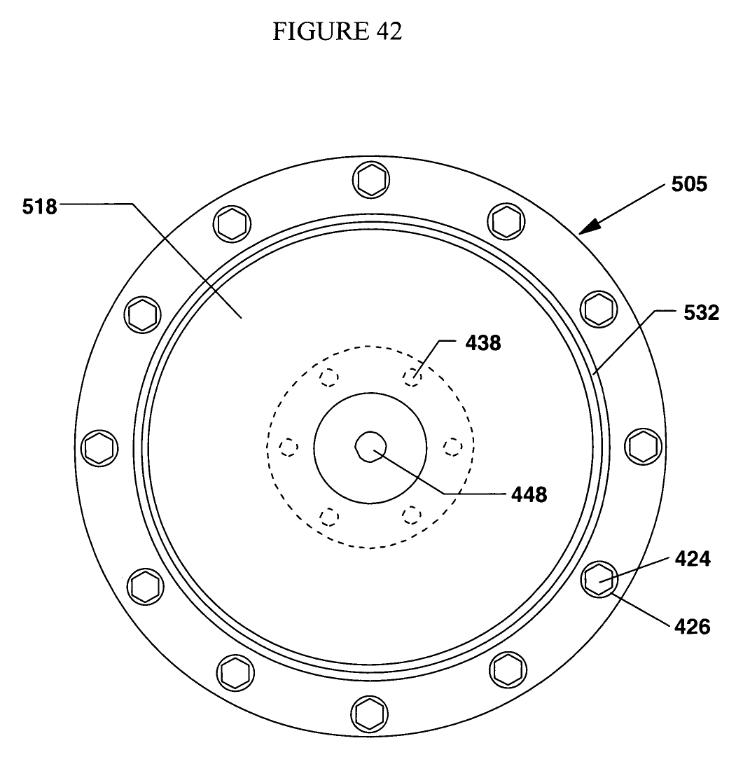

The rotor body 204 is then encased in a housing 214. The housing 214 is preferably made from materials sufficiently strong to withstand the hydraulic pressure contemplated in this invention. In one embodiment, shown in FIG. 25A, the housing 214 includes end plates 216, 218 mounted on the shaft 208 on either side of the rotor body 204. A cylindrical pipe 220 is positioned between the end plates 216, 218 and surrounds the rotor body 204. Studs and bolts or other fastening means 234 secure the end plates 216, 218 to each other. The force exerted by the connected end plates 216, 218 on the cylindrical pipe 220 holds the cylindrical pipe 220 in place. The end plates 216, 218, together with the cylindrical pipe 220, thereby form a hermetically-sealed housing 214 for the rotor body 204. A sealing means, such as an o-ring seal, may be located between the cylindrical pipe and the end plates and between the end plates and the shaft (236) to maintain pressure integrity within the housing 214 and minimize fluid leakage from the housing 214 into the atmosphere. While FIGS. 41-42 only illustrate one rotor body 204 encased in the housing 214, as increased volume or production capacity is needed, the distance between the end plates 216, 218 may be adjusted to accommodate additional rotor bodies 204 and thereby more chambers 200.

In an alternative embodiment, the housing 214 is a capsule-like structure formed preferably by two dome-like ends 222, 224 positioned around the rotor bodies 204 mounted on the shaft 208. The dome-like ends 222, 224 are secured to each other by, for example, bolts 238, to form a closed housing and sealing means 240 are preferably positioned at the interface of the dome-like ends and the shaft to maintain pressure integrity within the housing and minimize fluid leakage from the housing into the atmosphere. As shown, the housing 214 may encase multiple rotor bodies 204.

In the embodiments disclosed in FIGS. 25 and 25A, nutrient liquids and other fluids enter the housing through fluid input tubes 226 that penetrate the housing 214 to project the fluid into the chambers 200 of the rotor bodies 204. Proper sealing means are preferably used at the interface of the housing 214 and the fluid input tubes 226 and at the distal end 228 of the fluid input tubes 226 to maintain hermetical integrity.

As illustrated in FIGS. 25, 25A, a fluid output tube 230 is positioned along at least a portion of the length of the shaft 208. The fluid output tube 230 communicates with a liquid output mechanical end-face seal 27, as previously disclosed and described in relation to the embodiment of FIG. 10. Passages 232 connect the fluid output tube 230 to the chambers 200. Fluid from the chambers 200 travels along the passages 232 and is carried out of the housing 214 by the fluid output tube 230.

In use, small quantities of the living cells or subcellular biocatalysts in a nutrient medium are introduced through the fluid input tubes into the chambers and the housing. The rotating shaft rotates at a relatively low revolutions per minute (rpm) to stir the mixture and allow the cells to grow. The rotating shaft is then activated to rotate at a significantly higher rpm. The resultant increased centrifugal force forces the cells from the chambers and into the enclosed space of the housing. Nutrient liquids are then introduced into the chambers and the housing through the fluid input tubes and carry the cells back into the chamber. The inflow of nutrient fluid into the chamber counterbalances the centrifugal force exerted on the cells to capture and immobilize the cells within the chamber, while the liquid medium is able to flow out of the chamber through the fluid output tube of the rotating shaft.

The process of this invention is directed toward the immobilization of biocatalysts such as micro-organisms and eukaryotic cells, their subcellular organelles, and natural or artificial aggregates of such biocatalysts. Thus, the process system must be capable of immobilizing fairly light particles. It is known that the sedimentation rates of such particles due to gravity range from approximately 0.01 mm/min for small bacteria to 0.1 mm/min for small animal cells to more than 10.0 mm/min for thick-walled micro-organisms (such as yeasts) and biocatalytic aggregates such as bead-immobilized cells. We have analyzed the performance characteristics of the centrifugal bioreactor system of this invention using the dimensional configurations outlined above and present these results below.

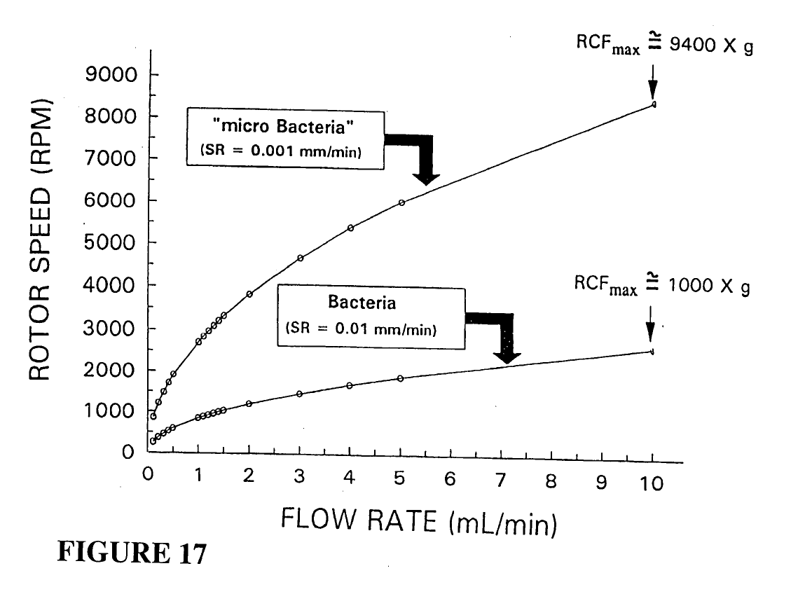

FIG. 17 displays profiles of the values of rotor speed and liquid flow rate which satisfy the boundary conditions outlined earlier for the rotor and bioreactor dimensions for typical biologically significant particles of the lowest two Sedimentation Rate (SR) ranges. The upper line displays the continuum of liquid flow rates and rotor speeds which result in the immobilization of particles of an intrinsic Sedimentation Rate (SR) of 0.001 mm/min, a value smaller by a factor of ten than any we have measured for any tested micro-organism. Note that, even at a flow rate of 10 mL/min, the rotor speed required to maintain immobilization is a physically reasonable value and that the maximum centrifugal force (RCF) required is approximately 9400×g, a value well within the physical limits of average quality centrifugal systems. The lower line displays the corresponding profile for particles of a Sedimentation Rate (SR) of 0.01 mm/min, a value near that exhibited by typical representative bacteria. Again, this line represents a continuum of values which satisfy the immobilization conditions. Thus for example, if a flow rate of 2.0 mL/min is required to adequately nutrition a particular sized three-dimensional array of “bacteria A,” a rotor speed near 1200 rpm will suffice, while a required flow rate of 8.0 mL/min necessitates a rotor speed near 2500 rpm. Note that the heavier particles of SR=0.01 mm/min require only a modest maximal centrifugal force of approximately 1000×g at a flow rate of 10.0 mL/min.

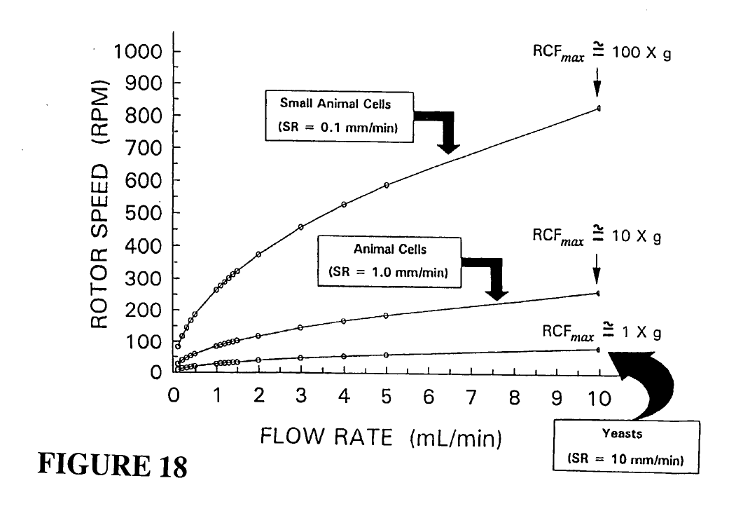

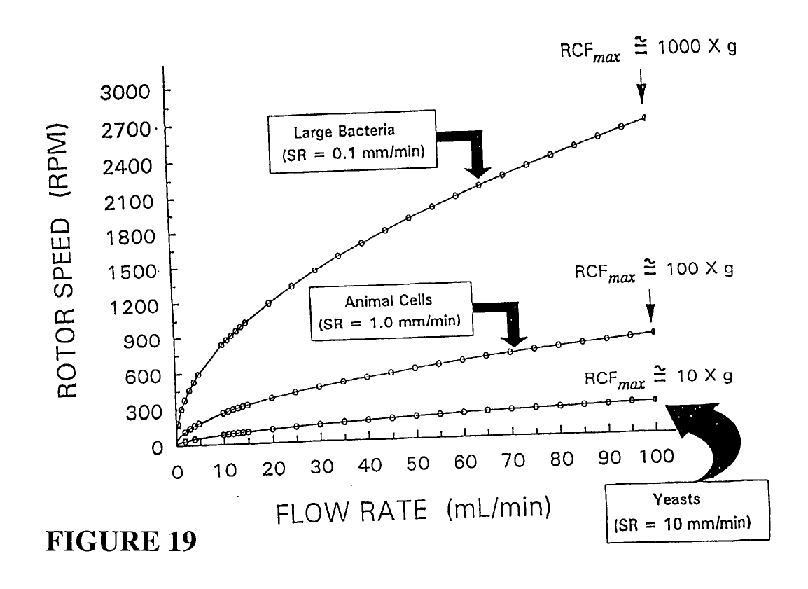

FIG. 18 displays profiles of the values of rotor speed and liquid flow rate which satisfy the boundary conditions outlined earlier for typical biologically significant particles of the higher three Sedimentation Rate (SR) ranges. The upper line displays the continuum of liquid flow rates and rotor speeds which result in the immobilization of particles comparable to larger micro-organisms or small animal cells (for example, mammalian erythrocytes) of an intrinsic Sedimentation Rate (SR) of 0.1 mm/min. The middle line displays the corresponding values for the immobilization of more typical animal cells (30 μm diameter; SR=1.0 mm/min), while the bottom line displays the continuum of values which provide for the immobilization of large dense cells, such as eukaryotic yeasts (SR=10 mm/min). As was the trend shown in FIG. 18, it is obvious from the data of FIG. 19 that the maximum rotor speeds and maximal centrifugal forces required in this flow rate range decrease as the intrinsic particle Sedimentation Rate (SR) due to gravity increases. Thus, for a flow rate of 10.0 mL/min, a three-dimensional array of average-sized animal cells requires only a relative centrifugal force of ca. 10×g to provide immobilization.

FIG. 19 displays profiles of the values of rotor speed and liquid flow rate which satisfy the boundary conditions outlined earlier for liquid flow rates of as much as 100 mL/min for the highest three intrinsic Sedimentation Rate (SR) ranges examined. Thus, even if the liquid flow rates required to nutrition such immobilized “beds” of particles (example bed volume=56 mL) is increased ten-fold, the maximal centrifugal forces and rotor speeds required are technically unremarkable. Note that, in the case of “animal cells” (SR=1.0 mm/min) a flow rate of 100 mL/min represents a flow of 6.0 L/hr, a flow obviously larger than that required to adequately nutrition such a three-dimensional array of cells under any imaginable conditions.

It is contemplated by the invention that the centrifugal fermentation device may be of any size, depending on the application desired. For example, the device may be three inches in diameter for small scale applications or may be six feet in diameter for large scale diameters. The invention contemplates all possible sizes for devices, and is not limited by these disclosed ranges.

The chamber caps may be attached by any methods known to those skilled in the art including, but not limited to, screw attachment. The chamber caps may or may not be detachable from the rest of the device. The shape of the chamber caps is determined by the particular application in which the device is employed. In a preferred embodiment, the chamber cap is a part of an assembly that screws into the cruciform structure. In an alternative embodiment, the chamber cap is made as one piece with the chamber.

The liquid inlet and outlet can be on the same side in a dual rotary seal, thus allowing direct drive on the opposite side trunnions or shaft. The fluid may also flow in pipes or conduits within the trunnions. The trunnion is preferably a driven trunnion that is driven by any means known to those skilled in the art, including but not limited to direct gearing or belts.

While it is generally obvious that the effect of immobilizing a population of, for example, bacteria in a flowing liquid will not lead to cellular damage as a result of the flow of liquid past the surface of such cells (since many micro-organisms possess extracellular “sheaths” which protect their plasma membranes from liquid shear forces), it is less obvious whether delicate animal cells (which do not possess such extracellular protection) would remain viable under these conditions. However, as was shown in FIG. 19, the maximum Relative Centrifugal Force (RCF) required to maintain an average-sized animal cell immobile in a liquid flow of 10 mL/min is 10×g. Even if this flow is raised to a level decidedly well above any anticipated nutritional need (100 mL/min), the maximum RCF required is only ca. 100×g (FIG. 19). It should be remembered that the immobilization of such a cell in a flowing liquid is the mathematical equivalent of moving the cell through a stationary liquid. Thus, since the conventional laboratory sedimentation of animal cells through liquid media at RCF's of more than 100×g is an unremarkable phenomenon, it is unlikely that the shear forces acting on such cells in the process of this invention will cause any damage to their plasma membranes. This assertion is supported by the operating characteristics of a related device, the Beckman JE-5.0 Centrifugal Elutriation System, from which viable animal cells have been successfully recovered after exposure to flow rates and RCF's greatly in excess of those proposed herein for the process of this invention.

With the invention, it is possible to immobilize three-dimensional arrays of biologically-significant particles and to adequately nutrition the immobilized particles with a completely liquid flow. In particular, for the small-scale prototypic centrifugal process outlined above, the required centrifugal forces and liquid flow rates are not unusual and present no novel problems such as, for example, requiring unreasonably high rotational speeds or flow rates. Further, it has been demonstrated that there is a wide range of paired flow rate and angular velocity values which maintain the immobilization of three-dimensional arrays of such particles.

The fact that there is a wide range of flow rates and corresponding rotational speeds which can be used to immobilize such arrays of particles has, however, a wider significance. Using conventional culture methodology, the major problem encountered in large-scale culture is the inability to adequately nutrition dense masses of metabolically-active biological units. In the case of conventional mammalian cell culture for example, an average cell density of more than 1×10⁶ cells/mL is rarely achieved for prolonged time periods for this reason. Similarly, bacterial cell densities between 1×10⁷ and 1×10⁹ cells/mL are rarely exceeded in mass culture by conventional methods for this same reason. Using the methodology of the process of this invention, as cell density and effective “bed” volume increases (either from cellular proliferation or bioreactor loading), the increased nutritional requirements of larger or more dense cultures can be met by increasing input liquid flow while simultaneously increasing the size of the applied centrifugal field. Using the process of this invention, it is possible to easily maintain mammalian cell cultures at concentrations two powers of ten greater than conventional, with bacterial cell densities approaching between 1×10¹⁰ and 1×10¹¹ cells/mL equally realizable.

Similarly, for dense cultures of aerobic organisms, the conventional problem of adequate delivery of optimal dissolved oxygen to the culture is solved using the process of this invention. Since it is possible to dissolve molecular oxygen in typical culture media at concentrations of more than 100 mM (using a hydraulic pressure of 1500 psig) the problem of the delivery of optimal dissolved oxygen, for any imaginable dense culture, is solved by adjusting the system hydraulic pressure to a value which will maintain the solubility of the desired concentration of oxygen. The ability to maintain dissolved oxygen concentration at optimal levels results in increased production efficiency. As has been noted by many researchers, the inability to achieve cellular production efficiencies near those observed in vivo is a major disadvantage of conventional animal culture techniques (The Scientist, 8, #22, pg. 16, Nov. 14, 1994).

The ability to achieve near-normal aerobic efficiency in dense culture has another, less obvious, advantage; the generation of heat. Instead of requiring expensive energy input to bring the liquid cellular environment to an optimal temperature, it is likely that the pumped liquid of the process of this invention will have to be delivered to the cellular environment at reduced temperatures to carry away excess metabolic heat.

Another advantage of the process of this invention is the relative invariance of the chemical composition of the liquid environment in which the three-dimensional arrays of biocatalysts are immobilized. Since the arrays are continually presented with fresh, optimal liquid nutrient input and since these arrays are continually drained by the continuance of the process flow, the chemical composition of the cellular environment will be completely invariant in time. There will be shallow chemical gradients of nutrients, product(s), and metabolites across the radial length of these arrays, but since the radial length is the shortest dimension of the array, these gradients will be minimal and can be easily compensated for by tailoring the media composition. Thus, for example, a pH change across the array depth can be compensated for with minimal buffering while input nutrient gradients across the array depth can be similarly compensated for.

Yet a further advantage of the process of the invention, however, is the fact that metabolic waste products will be continually removed from the cellular environments by the liquid process flow. Since it has been suggested that the inability to remove metabolic wastes and the inability to continually remove desired products from the cellular environment is a major factor in lowered per-cell productivity, it is likely that the utilization of the process of this invention will markedly increase general cellular productivity.

The chemical composition of optimal input liquid nutrient media to immobilized populations of biocatalysts in the process of this invention will be quite different from that of conventional nutrient media. In particular, the optimal media composition in this process will be that which can be completely consumed in one pass through the bioreactor chamber. Typical nutrient media contain a mix of as many as thirty or more nutrient chemicals, all of which are present in amounts which greatly exceed the nutritional needs of the biocatalysts. This is because the nutrient media must sustain their metabolic processes for as long as 100 hours in some cases. Similarly, conventional media contain concentrations of pH buffer compounds and indicators and hormonal stimuli (fetal sera and/or cytokines, etc.) in amounts which greatly exceed the immediate needs of the biocatalysts. In the process of this invention, the input liquid medium can be tailored to contain those concentrations of nutrients and stimulants which are directly required by the immediate metabolism of the immobilized biocatalysts. Ideally, the outflowing liquid which exits the bioreactor would be completely devoid of nutrients and contain only salts, metabolic wastes, and product molecules. The invention makes it possible to tailor the input media to maintain an immobilized cellular population in a nutritional state which either promotes or inhibits cellular proliferation. It is highly unlikely that a nutritional mix which is optimal for cellular division is optimal for the production of biochemicals by cells at rest in the cell cycle.

The liquid medium used in the invention may be any formulation known to those skilled in the art or may include specific individual components which are necessary for the biocatalyst of interest. The kinds of media may include, but are not limited to, a nutrient medium, a balanced salt solution, or a medium containing one or more organic solvents. The medium may contain dissolved gases for growth of the biocatalyst under anaerobic or aerobic conditions. The medium may be formulated so that the biocatalyst product or mobile biocatalysts found in the medium are more easily isolated.

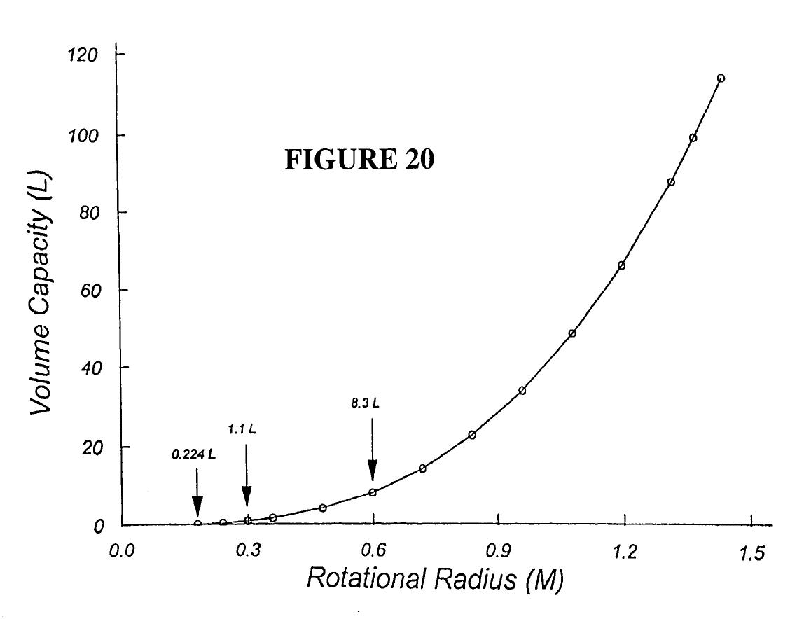

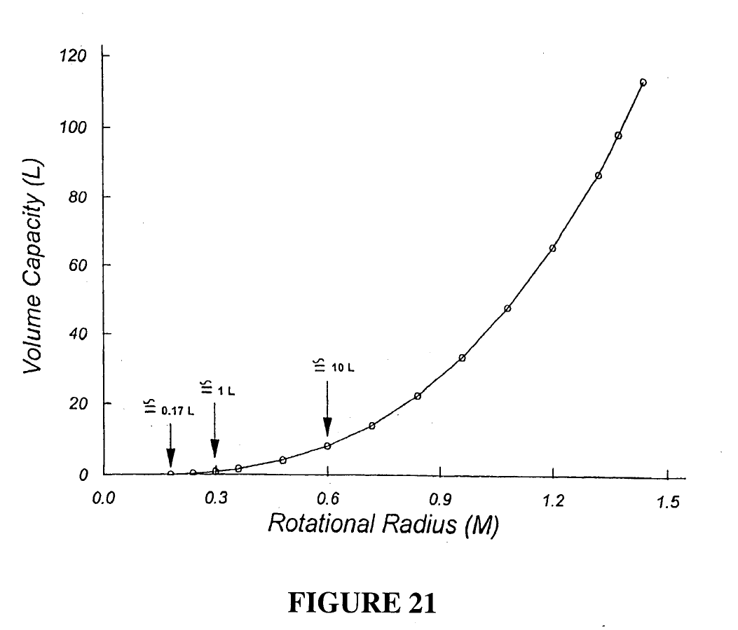

Another less obvious implication of the utility of this process methodology is the effect of scaling. In the first and second embodiments of the invention, the total volume capacity of the four-bioreactor rotor is approximately 224 mL and 170 mL, respectively. Note, however, that as the radius of the rotor is increased, the volume capacity of the system goes up as the cube of the radius. This is shown in the graph of FIG. 20, in which the leftmost point corresponds to the first embodiment of this invention, and in FIG. 21, in which the leftmost point corresponds to the second embodiment of this invention. A rotor with a radius of 1.5 meters would have a volume capacity of 120 liters. Further, since the average density of culture is roughly 100 times that of conventional culture methods, the equivalent culture volume is proportionally larger. Thus, a centrifugal fermentation unit with a rotor radius of 1.5 m is roughly equivalent to a 12,000 liter fermentation using current technology.

Finally, it should be noted that there is an additional advantage in scale in the use of the process of this invention. As a consequence of the fact that relative centrifugal force is directly proportional to the rotor radius but is also directly proportional to the square of the angular velocity, the rotational speeds required to maintain a desired relative centrifugal force decrease as the rotor radius is increased. This is shown graphically in FIG. 22. While the rotational speed required to maintain a RCF=100 g is approximately 810 rpm for a rotor with a radius of 18 cm, this required rotational speed drops to less than 300 rpm when the rotational radius is increased to 1.5 m. This is more than a 50% lowering in the speed of rotation.

While it is obvious that scale-up of this process will have value in industrial production facilities, it should be noted that a miniature embodiment of the Centrifugal Fermentation Process could be valuable in the analytical study of the “metabolic physiology” of small homogeneous populations of a particular cell type. For example, an exact nutritional requirements for maximal proliferation of, for example, a bacterial population are unknown—and could be rapidly and easily determined by perturbation of the composition of the nutritional liquid input to an immobilized test population while measuring some output parameter indicative of growth. Similarly, while it is desirable to know exactly what nutritional mix is optimal for cellular production of a biological product, such parameters are, again, unknown. Small-scale versions of the process of this invention could be advantageously utilized in advancing “analytical microbiology” or “analytical cell biology” in a fashion heretofore impossible to perform.

The invention may also be used for the continuous production of biological products which are secreted or otherwise released into the out-flowing liquid stream. Thus, for example, one might utilize this process for the continual harvest of product(s) which are released from an immobilized micro-organism population whose growth rate (and death rate) have been nutritionally manipulated to maintain a steady state immobilized “bed volume”. Such a process could run, theoretically, forever. Similarly, the immobilization of secretory animal cell populations would result in continual outflow of liquid enriched in the desired product(s).

The invention is also useful in the creation of non-secreted products (such as the cytosolic accumulation of protein in genetically-engineered E. coli). If an immobilized cell population is maintained in the bioreactor system outlined above, but under conditions of excess nutritional input, then the population will quickly grow to an enlarged bed size which will continually “leak” excess cells into the out-flowing liquid stream. Thus, the process of this invention can be operated as a “production cow”. That is, the invention can be used as a continual incubator for the production and outflow of mature cells which are rich in the desired product. Downstream isolation and disruption of the out-flowing cell stream to capture the product of interest would then follow conventional product purification methods.

The process of this invention offers the possibility of continual, serial interconversion of bio-organic substrates through several intermediate steps by two or more separate animal cell populations or micro-organism populations. As a consequence of the ability of the process of this invention to completely immobilize biocatalyst populations while continually flowing a liquid stream into and out of the immobilized population, it now becomes possible to serially connect separate, disparate immobilized populations into one flowing process stream with the assurance that there will be no cross-contamination of one population with the other. To accomplish this, several of the devices described herein are connected in series so that materials flow from one device into another device and then into the following device and so on. As is shown in FIG. 23, a process flow schematic in which a biochemical substrate, which is provided as a dissolved nutrient in the primary media reservoir, is converted into intermediate “product A” by its passage through the biocatalyst population immobilized in Centrifuge and Rotor #1 and is then further converted into “product B” by passage through a biocatalyst population immobilized in Centrifuge and Rotor #2. Furthermore, it is possible to change the composition of the liquid nutritional feedstock between the two immobilized populations since neither centrifuge/rotor combination is constrained to operate at the same flow rate and angular velocity as the other. Thus, as is shown in FIG. 23, the liquid flow into Centrifuge and Rotor #2 may be modified by means of an additional pump supplying necessary nutrients from Media Reservoir #2; the total flow per unit time through Centrifuge and Rotor #2 is simply higher than that through Centrifuge and Rotor #1.

A commercially-valuable example of the utility of a serial conversion process of this type is the biological production of acetic acid. Anaerobic bio-conversion of glucose into ethanol by an immobilized population of a yeast such as Saccharomyces cerevisiae in Centrifuge and Rotor #1 could be followed by aerobic conversion of ethanol to acetic acid by an immobilized population of the bacterium Acetobacter acetii located in Centrifuge and Rotor #2. This would require that dissolved oxygen and supplemental nutrients be provided via Media Reservoir #2 (using, for example, the oxygenation scheme depicted in FIG. 1).

Similarly, if a process flow scheme demanded that total flow volume per unit time through specific centrifugal bioreactor units be reduced, then a series of identical centrifugal bioreactor units could be connected in parallel to the process stream flow, with the resultant individual flow volume per unit time thereby reduced to the fractional flow through each unit. In this case, the devices of the invention would be connected in a parallel arrangement.

The microbial organisms which may be used in the invention include, but are not limited to, dried cells or wet cells harvested from broth by centrifugation or filtration. These microbial cells are classified into the following groups: bacteria, actinomycetes, fungi, yeast, and algae. Bacteria of the first group, belonging to Class Shizomycetes taxonomically, are Genera Pseudomonas, Acetobacter, Gluconobacter, Bacillus, Corynebacterium, Lactobacillus, Leuconostoc, Streptococcus, Clostridium, Brevibacterium, Arthrobacter, or Erwinia, etc. (see R. E. Buchran and N. E. Gibbons, Bergey's Manual of Determinative Bacteriology, 8th ed., (1974), Williams and Wilkins Co.). Actinomycetes of the second group, belonging to Class Shizomycetes taxonomically, are Genera Streptomyces, Nocardia, or Mycobacterium, etc. (see R. E. Buchran and N. E. Gibbons, Bergey's Manual of Determinative Bacteriology, 8th ed., (1974), Williams and Wilkins Co.). Fungi of the third group, belonging to Classes Phycomycetes, Ascomycetes, Fungi imperfecti, and Bacidiomycetes taxonomically, are Genera Mucor, Rhizopus, Aspergillus, Penicillium, Monascus, or Neurosporium, etc. (see J. A. von Ark, “The Genera of Fungi Sporulating in Pure Culture”, in Illustrated Genera of Imperfect Fungi, 3rd ed., V. von J. Cramer, H. L. Barnett, and B. B. Hunter, eds. (1970), Burgess Co.). Yeasts of the fourth group, belonging to Class Ascomycetes taxonomically, are Genera Saccharomyces, Zygosaccharomyces, Pichia, Hansenula, Candida, Torulopsis, Rhodotorula, Kloechera, etc. (see J. Lodder, The Yeasts: A Taxonomic Study, 2nd ed., (1970), North-Holland). Algae of the fifth group include green algae belonging to Genera Chlorella and Scedesmus and blue-green algae belonging to Genus Spirulina (see H. Tamiya, Studies on Microalgae and Photosynthetic Bacteria, (1963) Univ. Tokyo Press). It is to be understood that the foregoing listing of micro-organisms is meant to be merely representative of the types of micro-organisms that can be used in the fermentation process according to embodiments of the invention.

The culture process of the invention is also adaptable to plant or animal cells which can be grown either in monolayers or in suspension culture. The cell types include, but are not limited to, primary and secondary cell cultures, and diploid or heteroploid cell lines. Other cells which can be employed for the purpose of virus propagation and harvest are also suitable. Cells such as hybridomas, neoplastic cells, and transformed and untransformed cell lines are also suitable. Primary cultures taken from embryonic, adult, or tumorous tissues, as well as cells of established cell lines can also be employed. Examples of typical such cells include, but are not limited to, primary rhesus monkey kidney cells (MK-2), baby hamster kidney cells (BHK21), pig kidney cells (IBRS2), embryonic rabbit kidney cells, mouse embryo fibroblasts, mouse renal adenocarcinoma cells (RAG), mouse medullary tumor cells (MPC-11), mouse-mouse hybridoma cells (1-15 2F9), human diploid fibroblast cells (FS-4 or AG 1523), human liver adenocarcinoma cells (SK-HEP-1), normal human lymphocytic cells, normal human lung embryo fibroblasts (HEL 299), WI 38 or WI 26 human embryonic lung fibroblasts, HEP No. 2 human epidermoid carcinoma cells, HeLa cervical carcinoma cells, primary and secondary chick fibroblasts, and various cell types transformed with, for example, SV-40 or polyoma viruses (WI 38 VA 13, WI 26 VA 4, TCMK-1, SV3T3, etc.). Other suitable established cell lines employable in an embodiment of a method in accordance with the invention will be apparent to the person of ordinary skill in the art.

The products that can be obtained by practicing the invention are any metabolic product that is the result of the culturing of a cell, either eukaryotic or prokaryotic; a cell subcellular organelle or component, such as mitochondria, nuclei, lysozomes, endoplasmic reticulum, golgi bodies, peroxisomes, or plasma membranes or combinations thereof; or an enzyme complex, either a natural complex or a synthetic complex, i.e., a plurality of enzymes complexed together to obtain a desired product.

One of the advantages of the invention is the ability to produce a desired chemical from a cell without having to go through the laborious process of isolating the gene for the chemical and then inserting the gene into a suitable host cell, so that the cell (and thus the chemical) can be produced in commercial quantities. The invention may be used to directly culture, in high-density, a mammalian cell that is known to produce a desired chemical. By doing this, the invention may be used to produce large quantities of the desired chemical.

Products that can be produced according to embodiments of the invention include, but are not limited to, immunomodulators, such as interferons, interleukins, growth factors, such as erythropoietin; monoclonal antibodies; antibiotics from micro-organisms; coagulation proteins, such as Factor VIII; fibrinolytic proteins, such as tissue plasminogen activator and plasminogen activator inhibitors; angiogenic proteins; and hormones, such as growth hormone, prolactin, glucagon, and insulin.

Any culture medium known to be optimal for the culture of microorganisms, cells, or biocatalysts may be used in accordance with the embodiments of the invention. While such media are generally aqueous in nature for the culture of living organisms, organic solvents or miscible combinations of water and organic solvents, such as dimethylformamide, methanol, diethyl ether and the like, may be employed in those processes for which they are proved efficacious, such as those bioconversions in which immobilized biocatalysts are employed. Passage of the liquid media through the process system may be either one-pass or the liquid flow may be recycled through the system for higher efficiency of conversion of substrate to product. Desired nutrients and stimulatory chemicals may be introduced into the process flow, either via the low pressure nutrient supply or via injection into the process flow upstream of the cell chamber.

It will be appreciated that the invention is adaptable to any of the well-known tissue culture media including, but not limited to, Basal Medium Eagle's (BME), Eagle's Minimum Essential Medium (MEM), Dulbecco's Modified Eagle's Medium (DMEM), Ventrex Medium, Roswell Park Medium (RPMI 1640), Medium 199, Ham's F-10, Iscove's Modified Dulbecco Medium, phosphate buffered salts medium (PBS), and Earle's or Hank's Balanced Salt Solution (BSS) fortified with various nutrients. These are commercially-available tissue culture media and are described in detail by H. J. Morton (1970) In Vitro 6, 89-108. These conventional culture media contain known essential amino acids, mineral salts, vitamins, and carbohydrates. They are also frequently fortified with hormones such as insulin, and mammalian sera, including, but not limited to, bovine calf serum as well as bacteriostatic and fungistatic antibiotics.

Although cell growth or cell respiration within the biocatalyst immobilization chamber cannot be directly visualized, such metabolism may be readily monitored by the chemical sensing of substrate depletion, dissolved oxygen content, carbon dioxide production, or the like. Thus, for example in the case of a fermentation of a species of Saccharomyces cerevisiae, inoculation of the biocatalyst immobilization chamber with a small starter population of cells can be followed by an aerobic fermentation regime in which glucose depletion, dissolved oxygen depletion, and carbon dioxide production across the biocatalyst immobilization chamber are measured either chemically or via appropriate sensing electrodes. Thus, cell replication can be allowed to proceed until an optimal cell bed size is reached. Withdrawal of dissolved oxygen input at this time causes the immobilized yeast cells to shift into anaerobic fermentation of glucose with a resultant production of ethanol, a process which can likewise be monitored chemically.

Similarly, without any process modification, embodiments of the process in accordance with the invention can be utilized as a bioreactor for immobilized chemical catalysts, enzymes or enzyme systems. In such a process, a catalyst, an enzyme or an enzyme system is chemically immobilized on a solid support including, but not limited to, diatomaceous earth, silica, alumina, ceramic beads, charcoal, or polymeric or glass beads which are then introduced into the biocatalyst immobilization chamber. The reaction medium, either aqueous, organic, or mixed aqueous and organic solvents, flows through the process system and through the three-dimensional array of solid supports within the bioreactor. The catalyst, enzyme, or enzyme system converts a reactant in the process flow medium into the desired product or products. Similarly, in other applications, either cells or cell components including, but not limited to, vectors, plasmids, or nucleic acid sequences (RNA or DNA) or the like may be immobilized on a solid support matrix and confined for similar utilization in converting an introduced reactant into a desired product.

Commercial application of the invention can be in the production of medically-relevant, cellularly-derived molecules including, but not limited to, anti-tumor factors, hormones, therapeutic enzymes, viral antigens, antibiotics and interferons. Examples of possible product molecules which might be advantageously prepared using the method of the invention include, but are not limited to, bovine growth hormone, prolactin, and human growth hormone from pituitary cells, plasminogen activator from kidney cells, hepatitis-A antigen from cultured liver cells, viral vaccines and antibodies from hybridoma cells, insulin, angiogenisis factors, fibronectin, HCG, lymphokines, IgG, etc. Other products will be apparent to a person of ordinary skill in the art.

The increase in emitted greenhouse gases as a result of industrial growth and its putative effect on global warming is of worldwide concern. While many physical and chemical processes designed to remove gases from exhaust have been proposed, none are financially feasible. On the other hand microbial assimilation of aqueous gases, such as carbon dioxide, would be much cheaper and simpler than current remediation techniques, the central drawback to its usage has been the impossibility of economically processing large volumes. The high flow rates which would be required would “wash out” the desired microbial population well before the desired bioremediation is performed.

Microbial and algal populations are capable of direct assimilation of aqueous gases, such as carbon oxides (CO₂ and CO). Further, it has been amply demonstrated that virtually all terrestrial, as well as many marine microorganisms, exist in nature by attachment to a solid support through the agency of either homogeneous or heterogeneous biofilms. The invention comprises bioremediation processes that exploit these microbial characteristics to remove gases, such as carbon dioxide and monoxide, from gas sources, such as flue gas emissions, smokestacks and automobiles.

An embodiment of the invention comprises inert particles as the surface material. The microorganisms are added to the device, and there the microorganism attach to the inert particles. The microorganisms act upon the inert particles. This activity may cause chemical or physical changes, or both, to the inert particles. As a product of this activity, a metal is released. Preferably, the metal is not acted upon by the microorganism. Such metals include, but are not limited to, gold, platinum, copper and silver. Any metal, that is part of an ore composition, either chemically bound or physically trapped within the ore composition, is contemplated by the invention.

While it is known that microorganisms can act on inert particles to release metals, there has not been a process that easily allows for the growth and maintenance of such microbial colonies that are adequate to release efficient amounts of metal. The high flow rates that are required in some systems wash out the desired microbial population well before they can perform the desired activities. It is contemplated that the current invention includes this embodiment and all alterations in mechanical details that do not significantly alter the design. Minor modifications are included in this invention. Microorganisms include, but are not limited to, bacteria, viruses, fungi, algae, yeasts, protozoa, worms, spirochetes, single-celled and multi-celled organisms that are either procaryotes or eucaroytes that are known to those skilled in the art. Additionally, biocatalysts are included in this method.

In an embodiment, a microbial population, either homogeneous or heterogeneous, is immobilized on a solid support. Though not wishing to be bound by any particular theory, it is thought that such attachment is by the formation of biofilms. These solid supports are placed into a chamber, where the desired aqueous liquid flow is produced. The size and density of the solid support as well as the chamber dimensions are chosen to allow the system pump to achieve the desired throughput flow rate without the generation of excess liquid flow shear force on the immobilized biofilm. Since it is essential that the pumped system has only two phases (liquid and solid), the pumped system is maintained at hydraulic pressures above ambient by means of a pressure regulator, preferably downstream of the chamber. Nutrient minerals, organics, and dissolved gases are supplied to the chamber, under pressure, by a centrifugal fermentation unit (CBR) which also serves to re-charge the biofilm-immobilized microbial population with additional desired microbes. Where advantageous, input solution may be de-oxygenated by a gas sparging system available as a result of pressure release downstream of the output pressure regulator.

In a more preferred embodiment of the invention, the inert particles used as the surface material are made of iron pyrite, FeS₂. The iron pyrite ore is finely ground and added to the chamber. Bacteria that can metabolize the ore are added. In a preferred composition, the bacteria include various species selected from the Thiobacillis ferrioxidans sp. group. The bacteria initiate chemolithotropic processes which are oxygen dependent. Though not wishing to be bound by any particular theory, it is believed that the bacteria convert the FeS₂ into FeSO₄, ferrous sulfate. During this conversion, metals that are incorporated into the ore are released. One such preferred metal is gold. A constant slurry of ore is fed into the chamber to replenish the surface material that is being degraded or acted upon. The gold is easily retrieved from the chamber.

Use of other types of bacteria for isolation of metals is contemplated by the invention. The invention is not limited by the described microorganisms or surface materials. Any microorganisms capable of acting or degrading substrates that then release metals are contemplated by the invention.

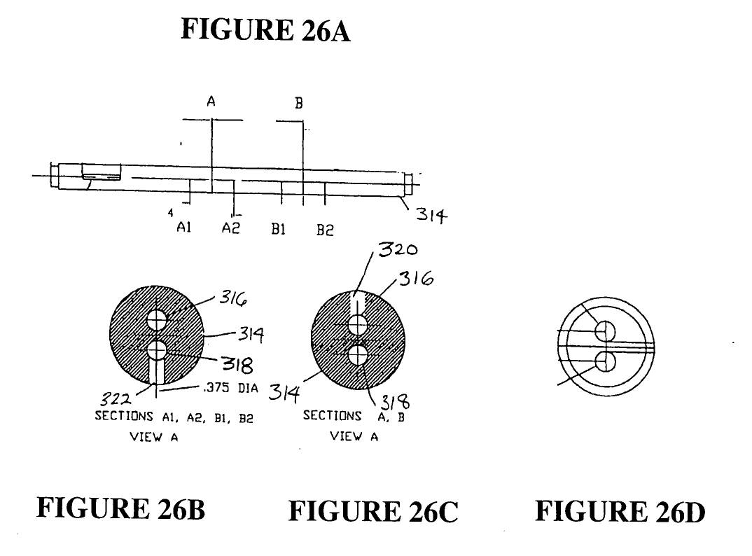

Another embodiment of the invention, as shown in FIGS. 25B and 25C, is directed to an apparatus for substantially immobilizing, containing, suspending and/or incubating a biocatalyst including a chamber system 310 having at least one chamber 312 for suspending the biocatalyst. In this embodiment, the chamber system 310 includes a plurality of chambers positioned along a longitudinal axis of a shaft. As depicted in FIG. 25C, five such chambers 312 are arranged along a shaft 314. However, the chamber system 310 may include any number of chambers. Turning to FIG. 26, the shaft 314 typically has an input cavity 316, an output cavity 318, an injection orifice 320, and an output orifice 322. The shaft 314 is typically composed of a stainless steel, typically 304 stainless steel annealed, ground, and polished. However, the shaft 314 may be composed of metals including, but not limited to, steel, iron, and titanium, plastics, composites, combinations thereof, or any material capable of withstanding stresses developed in the chamber system 310 during operation.

The input cavity 316 and the output cavity 318 preferably are positioned within the shaft 314 and extend throughout the length of the shaft 314. The injection orifice 320 and the output orifice 322 are in fluid communication with the input cavity 316 and the output cavity 318, respectively, and each orifice contacts an exterior surface of the shaft 316. The chamber system 310 further includes at least one injection element 324 which is in fluid communication with the injection orifice 320 and positioned within each chamber 312. Additionally, the chamber system 310 includes a means for rotating the shaft 314, such as a motor 326, and the at least one chamber 312 about the longitudinal axis of the shaft 314.

The chambers 312 typically include two sides 328, which may be composed of a material such as stainless steel. Alternatively, each side 328 may be composed of any material capable of withstanding the stresses developed during operation of the rotating chamber system 310, and may include, but is not limited to metals such as iron or titanium, plastics, composites and/or combinations thereof. Preferably, each chamber 312 possesses the shape of a triangular toroid, when viewed from the side. Specifically, the outermost portion of the chamber 312 maintains an angled portion 384 between each interior surface of the chamber 312 when viewed from a position generally orthogonal to the longitudinal axis of the shaft 314. The angled portion 384 may typically have an angle of about 50 to 60 degrees, and is preferably about 58 degrees. However, in another embodiment, the angled portion 384 may have an angle with a value greater than zero and less than 90 degrees, so long as a perimeter 376 of the chamber 312 is narrower in width than the width of the chamber nearest the shaft 314.

The angled portion 384 should be such that when the chamber system 310 is in operation, the biocatalyst 378 that is contained within each chamber 312 maintains a substantially stationary location which does not contact the exterior surface of a sleeve 340 or the shaft 314. The sleeve 340 is discussed in more detail below. Further, the chamber 312 should include a transition section or walls between the angled portion of the chamber and the sleeve or shaft. Typically, the transition section will be composed of a surface that is generally orthogonal to the longitudinal axis of the shaft 314. Positioning the transition section in this fashion discourages the biocatalyst 378 from contacting the sleeve 340 during operation of the chamber system 310 thereby allowing the biocatalyst 378 to grow and perform its intended function.

In another embodiment, the chamber 312 may include a compound triangular toroid shape to incubate two different types of biocatalysts having, for instance, different masses and/or sizes. Such a configuration may include a triangular toroid located at the perimeter of the chamber 312 and connected to a relatively larger triangular toroid located proximate to the sleeve 340. This compound shape may, for instance, allow one biocatalyst to be immobilized proximate to the outer toroid and another biocatalyst to be immobilized proximate to the inner toroid. Further, it should be understood that the shape of the chamber 312 may include any shape which may be calculated using the equations and processes set forth in U.S. Pat. No. 5,821,116.

In one embodiment, the diameter of a chamber 312 may include dimensions within the range of from about 20 to 30 inches (50.8 to 76.2 cm), and preferably about 26 inches (66 cm). However, in another embodiment, the diameter of the chamber 312 may include dimensions within the range from about 48 to 60 inches (121.9 to 152.4 cm). The sides 328 of the chamber 312 are typically fastened together using a plurality of bolts 330. The bolts 330 are positioned within the holes 332 located around the perimeter 376 of the chamber 312. Alternatively, each side 328 of the chamber 312 may be held together using any assortment of fasteners or other releasable connection mechanisms. Once the sides 328 of the chamber 312 have been assembled together, the interior surfaces of each side 328 of the chamber may be within the range of about 4 to 12 inches (10.2 to 30.5 cm) apart at the point nearest the sleeve 340 and preferably eight inches. However, other embodiments of the chamber system 310 may include a chamber 312 having a width greater than 12 inches (30.5 cm) in accordance with the spirit of this invention and U.S. Pat. No. 5,821,116.

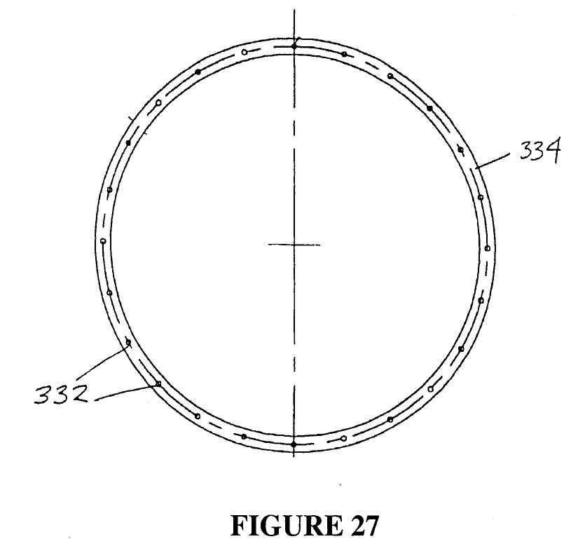

The mechanical connection may be further strengthened by locating a reinforcement ring 334, as shown in detail in FIG. 27, between the exterior surface of a flange 336 of each side 328 of the chamber 312. While the ring 334 is preferably constructed of aluminum, it may also include materials such as, but not limited to, stainless steel or plastic. Further, the reinforcement ring 334 need not be included within the chamber system 310, if each side 328 of the chamber 312 is constructed of stainless steel or other material capable of withstanding the stresses to which the chamber system 310 is subjected. A seal between each side 328 may be established using an o-ring 333, not shown, which is typically positioned on the interior surface of the flange 336 of the chamber 312. Thus, the o-ring 333 is located in a recessed portion of the flange 336, not shown, to position the o-ring 333. Once the sides 328 are assembled, the o-ring 333 contacts both sides 328. Alternatively, the seal between each side 328 of a chamber 312 may be created using means including, but not limited to, a releasable adhesive, a gasket or any type of sealant material.

The motor 326 (see FIGS. 25B-C) of the chamber system 310 is mounted to a stand 338 and is connected to the shaft 314 via a pulley belt 368 and a drive pulley 370. The drive pulley 370 is mechanically fastened to the shaft 314, preferably using a weld, an adhesive, a keyway, or other mechanical-type connection. The stand 338 positions the shaft 314 perpendicular to a gravitational force which is typically accomplished by locating the shaft parallel to the Earth's surface. The stand 338 includes two sets of legs 372 which are designed to restrict the shaft 314 from any movement, except rotational movement, about the longitudinal axis of the shaft 314. The stand 338 includes bearing assemblies 374 which allow the shaft 314 to rotate while maintaining its position. In operation, the motor 326 is used to rotate the shaft 314 and the plurality of chambers 312 attached thereto about the longitudinal axis of the shaft 314. The motor 326 is capable of rotating the shaft 312 at any desired rate.

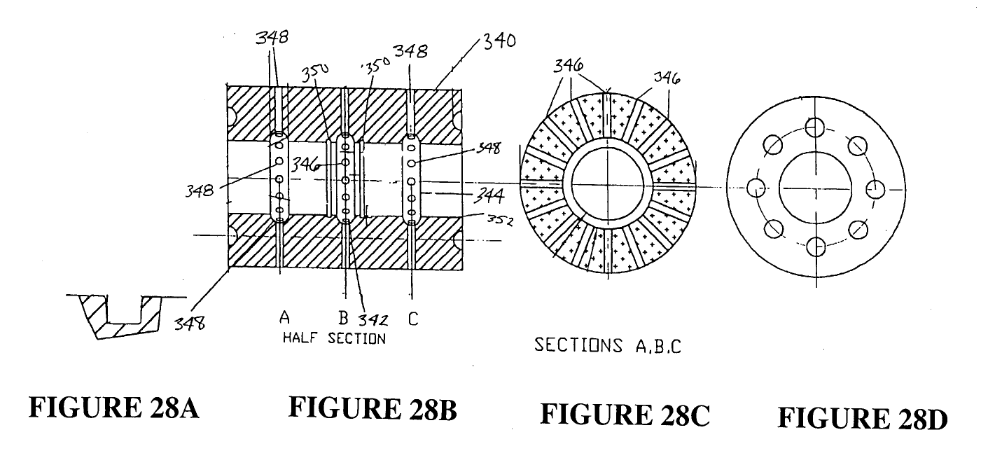

Referring now to FIG. 28A-D, each chamber 312 may include a sleeve 340 having an input channel 342, at least one output channel 344 in an inner wall of the sleeve 340 and a plurality of input apertures 346 and a plurality of output apertures 348 extending between the input channels 342 and output channels 344 respectively and an outer wall of the sleeve 340. Preferably, the input channel 342 is positioned at a midpoint of a longitudinal axis of the sleeve 340. Alternatively, the input channel 342 may be positioned at any point along the longitudinal axis of the sleeve 340. In the preferred embodiment, the input channel 342 is positioned between two output channels 344. O-rings 350, shown in FIG. 28B, are typically located within the inner wall of the sleeve 340 and are positioned between each output channel 344 and the input channel 342. Alternatively, o-rings 350 may be positioned on the shaft 314. The o-rings 350 provide a seal to prohibit fluid flow between the sleeve 340 and the shaft 314. Further, the o-rings 350 allow the sleeve 340 to be removed from the shaft 314.

The sleeve 340 is positioned on the shaft 314 so that the input channel 342 is in fluid communication with the injection orifice 320 of the shaft 314, and each output channel 344 is in fluid communication with each output orifice 322 located within the shaft 312. In such a position, the o-rings 350 located between the input channel 342 and each output channel 344 form a seal between the sleeve 340 and the shaft 314 which prevents the input fluid from mixing with and contaminating the output fluid. The plurality of input apertures 346 extend from the input channel 342 to the outer wall of the sleeve 340. Similarly, the plurality of output apertures 348 extend from the output channel 344 to the outer wall of the sleeve 340.

The sleeve 340 is sized to fit completely within the chamber 312 once both sides 328 of the chamber 312 have been fastened together. An internal diameter 352 of the sleeve 340 is typically slightly larger than an outside diameter of the shaft 314. The sleeve 340 is capable of being positioned on the shaft 314 by sliding the sleeve 340 on the shaft 314. Of course, there are numerous ways to position the sleeve 340 to the shaft 314 as would be understood by one of ordinary skill in the art including using a press fit or other mechanical connection. Further, the internal diameter 352 of the sleeve 314 is sized to allow the o-rings 350 to properly seat within a recessed bed, not shown, and against the exterior surface of the shaft 314.

The sleeve 340 is positioned within the chamber 312 to reduce the volume of liquid which is located between the biocatalyst 378 and the shaft 314 and which provides little, if any, benefit to the biocatalyst 378. If the sleeve 340 were not present, the volume of liquid located between the shaft 314 and the biocatalyst 378 would cause an increase in the stresses imparted on the perimeter 376 of the chamber 312. Positioning the sleeve 340 within the chamber 312 reduces the volume of liquid capable of filling the chamber 312 during operation. Thus, it is theorized, without wishing to be bound by the theory, that the stresses produced during operation of the chamber system 310 using the sleeve 340 are less than the stresses produced during operation of a chamber system 310 without a sleeve 340.

Alternatively, the same result may be achieved by increasing the diameter of the shaft 314 to produce a reduced volume within the chamber 312, thereby eliminating the need for the sleeve 340. In this embodiment, the shaft 314 includes a plurality of injection orifices 320 in fluid communication with the input cavity 316 and arranged in a pattern similar to the pattern of input apertures 346 of the sleeve 340. As a result, the plurality of injection elements 324 are connected directly to the plurality of injection orifices 320 rather than to the plurality of input apertures 346. Further, the shaft 314 of this embodiment includes a plurality of output orifices 322 capable of receiving output fluid. The plurality of output orifices 322 are similarly arranged to the orifices on the sleeve 314.

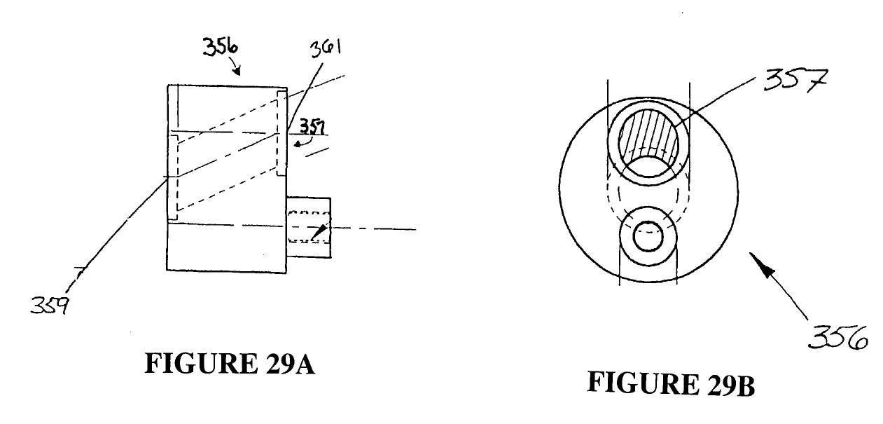

It will be understood by one of ordinary skill in the art that there is more than one way to accomplish placing the chamber system 310 in fluid communication with a source of input fluid. Referring again to FIG. 25B, the chamber system 310 may further include an input feed tube 354 that is in fluid communication with the input cavity 316, as shown herein, using a flow diverter 356 and as further shown in detail in FIG. 29. The flow diverter 356 includes a bore 357 having a first center point 359 at one end of the flow diverter 356 and extending to the other end of the flow diverter 356, where a second center point 361 of the bore is off-center from the longitudinal axis a distance equal to the distance which the input cavity 316 is off-center from the longitudinal axis of the shaft 314. The flow diverter 356 connects the input feed tube 354 in fluid communication with the input cavity 316. In a similar fashion, the output cavity 318 is placed in fluid communication with the output tube 358 through the use of a second fluid diverter 363, as illustrated in FIG. 25B. The output tube 358 is used to remove the fluid exiting the chamber system 310 and to deposit it in a holding tank, reservoir, or other location.

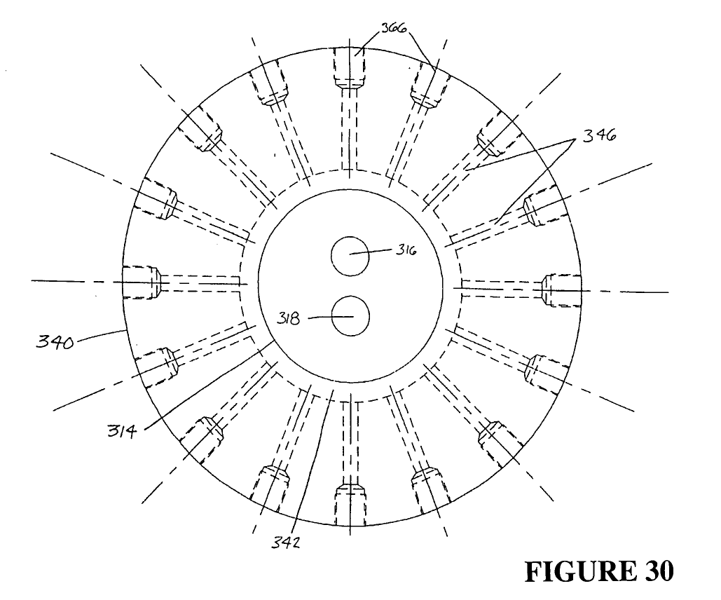

It will be understood by one of ordinary skill in the art that there is more than one way to accomplish injecting the input fluid proximate to the perimeter 376 of the chamber 312. Referring now to FIG. 30, the chamber system 310, as shown herein, includes a plurality of injection elements 324 in fluid communication with the plurality of input apertures 346 of the sleeve 340. Each injection element 324 is connected to the sleeve 340 and extends outwardly toward the perimeter 376 of the chamber 312 in a direction generally orthogonal to the longitudinal axis of the sleeve 340. Each injection element 324 typically includes a threaded fitting 360 at one end of an extension arm 362 and a nozzle 364 at the opposite end of the extension arm 362. Each threaded fitting 360 is connected to an input aperture 346 of the sleeve 340 using, for instance, a recessed receiver 366, as shown in FIG. 30. Each extension arm 362 is sized to position each nozzle 364 proximate to the perimeter 376 of the chamber 312. Preferably, the nozzles 364 are composed of precise orifices which produce a relatively even liquid stream. The precise orifices further enable the nozzles 364 to be used to evenly distribute input fluid to the perimeter 376 of the chamber 312.

In one embodiment, the plurality of nozzles 364 do not contact the interior surface of the chamber 312. In this embodiment, the extension arms 362 are constructed of materials capable of withstanding the forces generated by the system 310 during operation. In another embodiment, the plurality of nozzles 364 contact the interior surface at the perimeter 376 of the chamber 312 in a manner enabling fluid to be released at the perimeter 376.

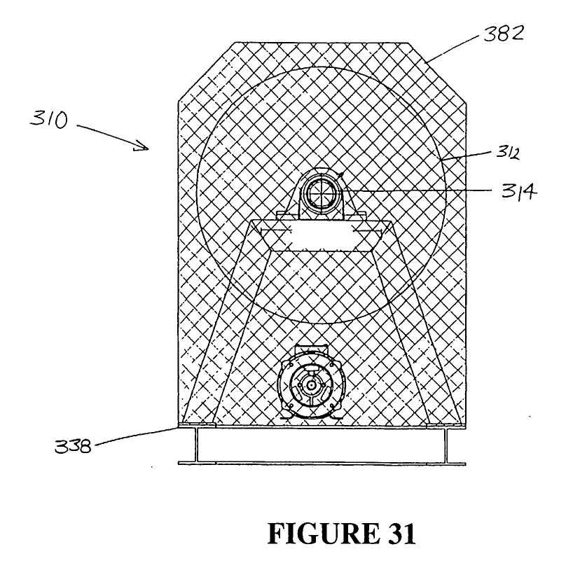

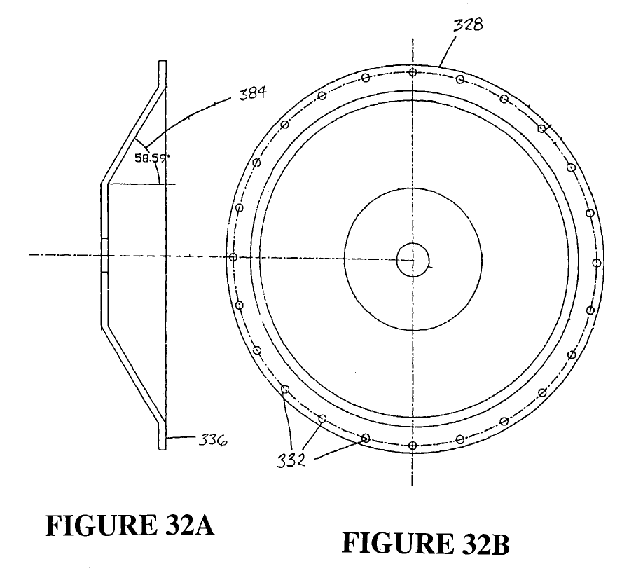

In operation, the chamber 312 houses a biocatalyst 378, positioned between the exterior surface of the sleeve 340 and the interior surface of the chamber 312. The motor 326, together with the pulley belt 368 and drive pulley 370, rotate the shaft 314 and at least one chamber 312 at a desired rate. The chamber system 310 typically includes a shield 382, as shown in FIGS. 31 and 32, which may include two halves and may be hinged at opposing ends of the stand 338 to allow for easy removal of the shield. The shield 382 protects individuals from contacting the rotating chambers 312. As the chamber 312 is rotated, pressurized fluid is typically delivered to each chamber 312 via the input feed tube 354, the input cavity 316, the injection orifice 364, the plurality of input apertures 346, and the plurality of injection elements 324. The pressure of the fluid may be monitored using a pressure gauge 380. The injection elements 324 release the pressurized fluid proximate to the interior surface of the perimeter 376 of the chamber 312 preferably located the furthest distance from the longitudinal axis of the shaft 314.

After the fluid has been released, the fluid flows from the outermost portion of the chamber 312 inwardly toward the plurality of output apertures 348 located on the exterior surface of the sleeve 340. When the design of the chamber 312 is a triangular toroid, as set forth above, the fluid injected into the chamber 312 decreases in velocity as it moves from the perimeter 376 of the chamber 312 inwardly toward the longitudinal axis of the shaft 314. The velocity of the fluid is reduced because the cross-sectional area of the chamber 312 increases in size moving from the perimeter 376 of the chamber 312 toward the longitudinal axis of the shaft 314. Injecting the pressurized fluid at the perimeter 376 of the chamber 312 positions the fluid so that it must diffuse through the biocatalyst 378 before it leaves the system 310 via the plurality of output apertures 348.

The chamber system 310 positions and incubates a biocatalyst 378 for various beneficial purposes. The chamber system 310 may include a biocatalyst 378, for example, comprised of biocatalysts capable of removing contaminants and heavy metals from wastewater. In such an application, the biocatalyst 378 is used to cleanse water by removing harmful materials. In another application, the biocatalyst 378 is composed of mammalian cells which are used to produce a variety of beneficial materials including, but not limited to, enzymes. Furthermore, in another application, the biocatalyst 378 is composed of mammalian cells which are used to produce monoclonal antibodies. Examples of containment of or immobilization of a biocatalyst are given herein and in U.S. Pat. No. 5,821,116.

As mentioned above, the chamber system 310 may include five chambers 312 located adjacent one another on a single shaft 314, as shown in FIGS. 25 and 25A-C. In this embodiment, when for instance, the biocatalyst 378 is being utilized to remove contaminants from a wastewater stream, (the input fluid), each chamber 312 is capable of producing about 90 gallons of output (“clean” effluent) per 24 hours to about 120 gallons of output per 24 hours (about 341 to 454 liters per 24 hours) using the parameters disclosed herein and, for instance, a Pseudomonas putida bacteria as the biocatalyst. However, this amount may vary based upon the characteristics of the cells forming the biocatalyst. For instance, a biocatalyst composed of yeast is capable of producing about 900 to 1,200 gallons per 24 hours (about 3,406 to 4,543 liters per 24 hours) because the yeast are about 10 times heavier than the Pseudomonas putida bacteria. Thus, the total output for a five chamber system can be between about 450 and 600 gallons per 24 hours (about 1,703 to 2,271 liters per 24 hours) using the Pseudomonas putida bacteria.

However, the chambers 312 may be increased in diameter, while maintaining their triangular toroidal shape, to treat a larger quantity of input fluid and produce a larger quantity of output fluid. In this embodiment, the chamber 312 may have a diameter of about 4.5 feet (137 cm). In this embodiment, each chamber 312 has the capacity of about 1,800 gallons of output per 24 hours (about 6,813 liters per 24 hours). Thus, the alternative five chamber system has the ability to produce about 9,000 gallons of output per 24 hours (about 34,069 liters per 24 hours).

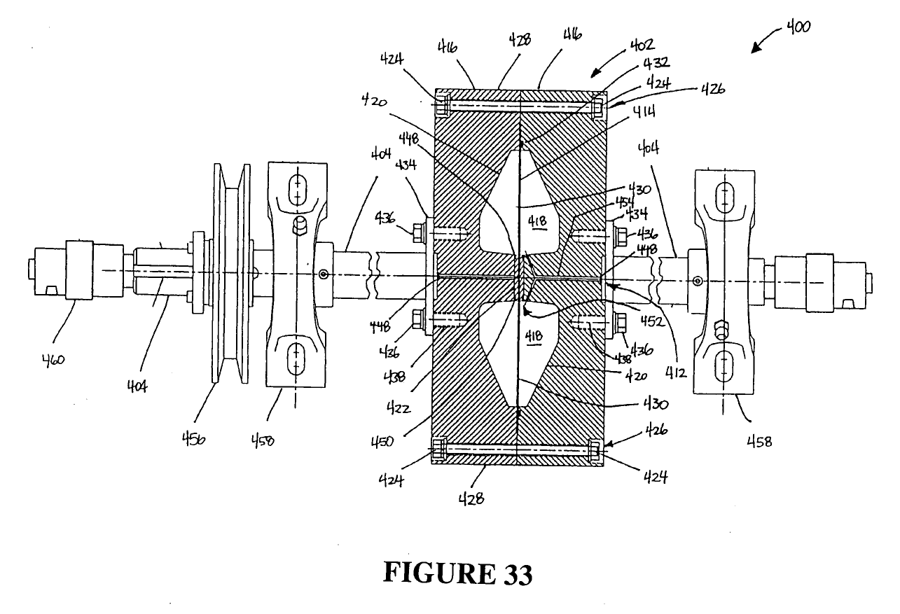

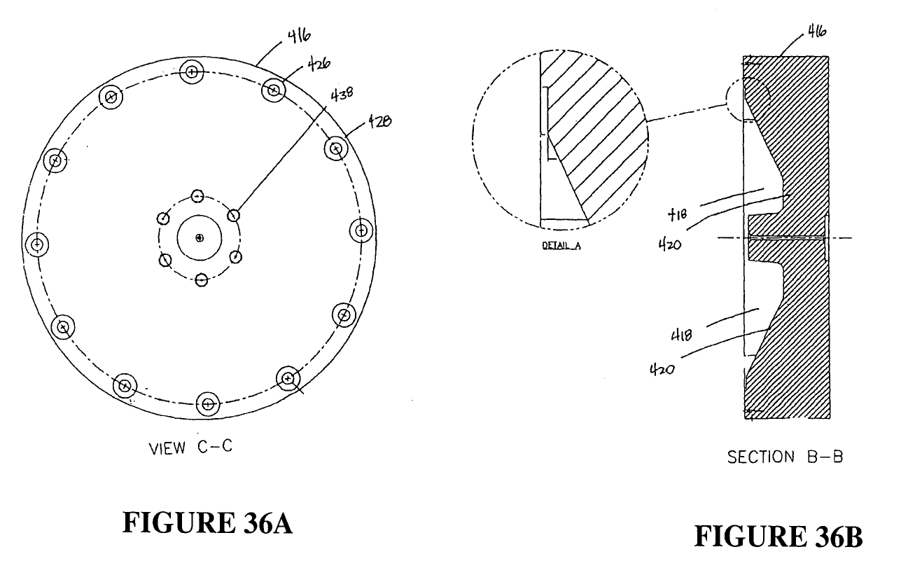

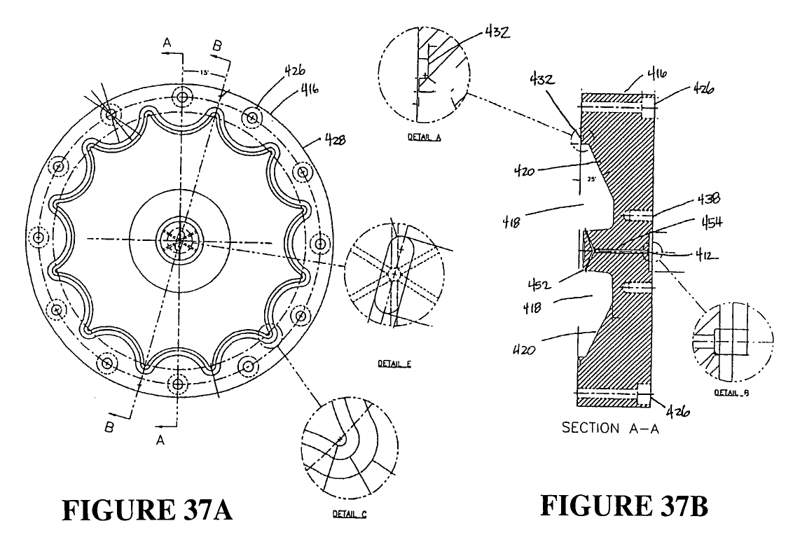

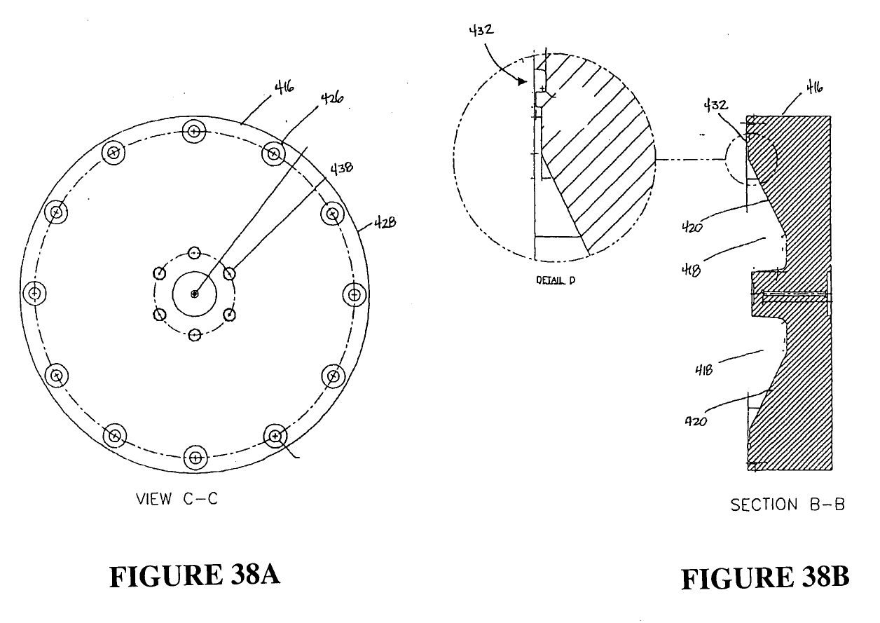

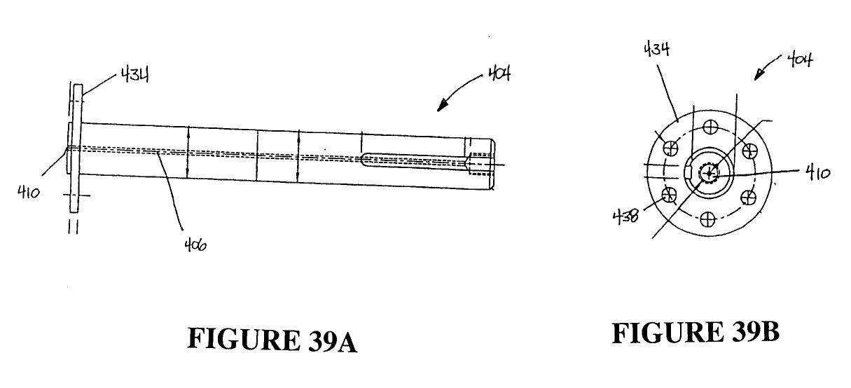

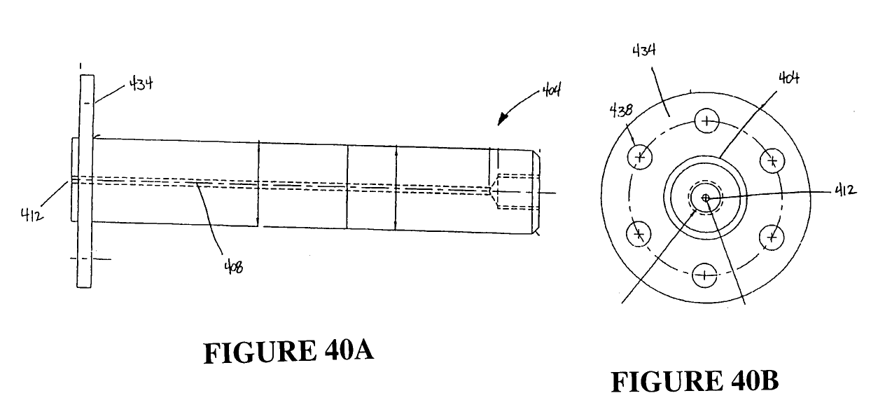

In another embodiment of the invention, a chamber system is illustrated in FIGS. 33-41 that improves upon sealing between various parts of the chamber system and improves fluid flow and circulation within the chamber system. Furthermore, the embodiment of the chamber system described in FIGS. 33-41 is relatively easier to clean and sterilize. The embodiment shown also operates in accordance with the invention as described in the embodiments above.

FIG. 33 is a side view of a chamber system according to another embodiment of the invention. This embodiment is also directed to an apparatus for substantially immobilizing, containing, suspending and/or incubating a biocatalyst including a chamber system 400 having at least one chamber 402 for suspending a biocatalyst. In this embodiment, the chamber system 400 includes at least one chamber 402 positioned along a longitudinal axis of a shaft 404. Note that the chamber system 400 may include any number of chambers 402 mounted to the shaft 404. Turning to FIGS. 81 and 82, the shaft 404 typically has an input cavity 406, an output cavity 408, an injection orifice 410, and an output orifice 412. The shaft 404 is typically composed of a stainless steel, typically 304 or 316 stainless steel annealed, ground and polished. However, the shaft 404 may be composed of metals including, but not limited to, steel, iron, and titanium, plastics, composites, combinations thereof, or any material capable of withstanding stresses developed in the chamber system 400 during operation.

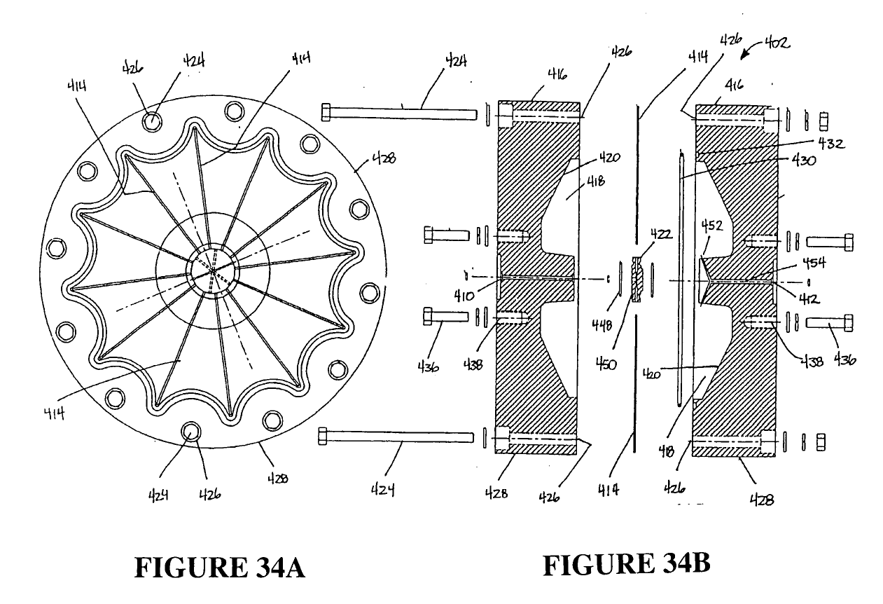

The input cavity 406 and the output cavity 408 preferably are positioned within the shaft 404 and extend throughout the length of the shaft 404. The injection orifice 410 and the output orifice 412 are in fluid communication with the input cavity 408 and the output cavity 410, respectively, and each orifice contacts an exterior surface of the shaft 406. The chamber system 400 further includes at least one injection element 414 which is in fluid communication with the injection orifice 410 and positioned within each chamber 402, as shown in FIGS. 33 and 34A-B. In this embodiment, a plurality of injection elements 414 are shown in FIG. 34A. Additionally, the chamber system 400 includes a means for rotating the shaft 404, such as a motor (similar to 326 in FIG. 25), and at least one chamber 402 about the longitudinal axis of the shaft 404.

As shown in FIGS. 33-38, a chamber 402 typically includes two sides 416, which may be composed of a material such as stainless steel. Alternatively, each side 416 may be composed of any material capable of withstanding the stresses developed during operation of the rotating chamber system 400, and may include, but is not limited to metals such as iron or titanium, plastics, composites and/or combinations thereof. Note that in this embodiment, when the sides 416 of the chamber 402 are fit together that each chamber 402 has an internal cavity 418 in the shape of a triangular toroid when viewed from the side. Furthermore, the external shape of the chamber 402 is desirably round and wheel-shaped when the two sides 416 are fit together. The outermost portion of the internal cavity 418 maintains an angled portion 420 between each interior surface of the chamber 402 when viewed from a position generally orthogonal to the longitudinal axis of the shaft 404. The angled portion 420 may typically have an angle of about 0 to 90 degrees, and is preferably about 25 degrees.

The angled portion 420 should be such that when the chamber system 400 is in operation, the biocatalyst that is contained within the chamber 402 forms a substantially stationary biocatalyst which does not contact the exterior surface of a manifold sleeve 422 or the shaft 404. (The manifold sleeve 422 is discussed in more detail below.) Further, the chamber 402 should include a transition section or walls between the angled portion of the chamber and the sleeve or shaft. Typically, the transition section will be composed of a surface that is generally orthogonal to the longitudinal axis of the shaft 404. Positioning the transition section in this fashion discourages the biocatalyst from contacting the manifold sleeve 422 during operation of the chamber system 400 thereby allowing the biocatalyst to grow and perform its intended function.

The sides 416 of the chamber 402 are typically fastened together using a plurality of bolts 424. The bolts 424 are positioned within the holes 426 located around the perimeter 428 of the chamber 402. Alternatively, each side 416 of the chamber 402 may be held together using any assortment of fasteners or other releasable connection mechanisms. Once the sides 416 of the chamber 402 have been assembled together, the width of the internal cavity 418 of the chamber 402 may be approximately 2.6 inches (6.7 cm) with the diameter of the chamber being approximately 12.0 inches (30.5 cm), and the diameter as measured between opposing bolts 424 is approximately 9.8 inches (25.0 cm). However, other embodiments of the chamber system 400 may include a chamber 402 having dimensions in accordance with the spirit of this invention and in U.S. Pat. No. 5,821,116.

A seal between each side 416 may be established using an o-ring 430, which is typically positioned on the interior surface of a recessed portion 432 of the internal cavity 418 of the chamber 402. Once the sides 416 are assembled, the o-ring 430 contacts both sides 416. Alternatively, the seal between each side 416 of a chamber 402 may be created using means including, but not limited to, a releasable adhesive, a gasket or any type of sealant material.

As shown in FIGS. 33, 39, and 40, the shaft 404 can be divided into two portions that each mount to the chamber 402 at or near the central portion of a respective side 416 of the chamber 402. A flange 434 on each portion of the shaft 404 permits the shaft 404 to connect to the exterior surface of the chamber 402. Bolts 436 are positioned within holes 438 located in the flange 434 and machined into the exterior surface of the chamber 402. Alternatively, the shaft 404 may be secured proximate to the chamber 402 using any assortment of fasteners or other releasable connection mechanisms. Once the shaft 404 has been connected to the sides 416 of the chamber 402, the shaft 404 can then be driven to rotate the shaft 404 which transmits its rotational force through the flange 434 and to the chamber 402.

Referring now to FIG. 41, the chamber 402 may include a manifold sleeve 422 having an input channel 440, at least one output channel 442 in an inner wall of the manifold sleeve 422 and a plurality of input apertures 444 and a plurality of output apertures 446 extending between the input channels 440 and output channels 442 respectively and an outer wall of the manifold sleeve 422. Preferably, the input channel 440 is positioned at a midpoint of a longitudinal axis of the manifold sleeve 422. Alternatively, the input channel 440 may be positioned at any point along the longitudinal axis of the manifold sleeve 422. In the preferred embodiment, the input channel 440 is positioned between a plurality of output channels 442. O-rings 448, shown in FIGS. 33 and 34, are typically located at a recessed edge 450 along the outer wall of the manifold sleeve 422. Furthermore, O-rings 448 may be positioned adjacent to the shaft 402 and around the injection orifice 410 and the output orifice 412. The O-rings 448 provide a seal to prohibit fluid flow between the shaft 404 and the chamber 402.

The manifold sleeve 422 is positioned in the chamber 402 so that the input channel 440 of the manifold sleeve 422 is in fluid communication with the injection orifice 410 of the shaft 404, and each output channel 442 of the manifold sleeve 422 is in fluid communication with each output orifice 412 located within the shaft 404. In such a position, the o-rings 448 located between the manifold sleeve and the sides 416 of the chamber 402 form a seal which prevents the input fluid from mixing with and contaminating the output fluid. The plurality of input apertures 444 extend from the input channel 440 to the outer wall of the manifold sleeve 422. Similarly, the plurality of output apertures 446 extend from the output channel 442 to the outer wall of the manifold sleeve 422.

The manifold sleeve 422 is sized to fit completely within the chamber 402 once both sides 416 of the chamber 402 have been fastened together. Of course, there are numerous ways to position the manifold sleeve 422 relative or proximate to the shaft 404 as would be understood by one of ordinary skill in the art. Furthermore, it will be understood by one of ordinary skill in the art that there is more than one way to accomplish placing the chamber system 400 in fluid communication with a source of input fluid.

From the internal cavity 418 of the chamber 402, as shown in FIGS. 33, 34, and 37, fluid can exit the chamber 402 via a plurality of chamber output apertures 452. From these apertures 452, fluid travels through a chamber output channel 454 to the output aperture 412 and then through the shaft 404 via the outlet cavity 408, where the fluid can be collected from the system 400.

The motor of the chamber system 400 mounts to a stand (similar to 338 in FIG. 25) and is connected to the shaft 404 via a pulley belt (similar to 368 in FIG. 25) and a drive pulley 456. The drive pulley 456 is mechanically fastened to the shaft 404, preferably using a weld, an adhesive, a keyway, or other mechanical-type connection. The stand positions the shaft 404 perpendicular to a gravitational force which is typically accomplished by locating the shaft 404 parallel to the Earth's surface. As previously described in FIG. 25, the stand is designed to restrict the shaft 404 from any movement, except rotational movement, about the longitudinal axis of the shaft 404. The stand mounts to bearing assemblies 458 which allow the shaft 404 to rotate while maintaining its position. In operation, the motor is used to rotate the shaft 404 and one or more chambers 402 attached thereto about the longitudinal axis of the shaft 404. The motor is capable of rotating the shaft 404 at any rate desired by the user.

In operation, the chamber 400 houses a biocatalyst positioned between the exterior surface of the manifold sleeve 422 and the interior surface of the chamber 402. The motor, together with the pulley belt and drive pulley 456, rotate the shaft 404 and at least one chamber 402 at a desired rate. The chamber system 400 typically includes a shield or safety containment chamber, which may include two halves and may be hinged or bolted at opposing ends of the stand to allow for easy removal of the shield or safety containment chamber. The shield or safety containment chamber provides a thermal barrier or heat containment device for maintaining a constant temperature inside the shield or safety containment chamber where the chamber 402 contains the biocatalyst. Furthermore, the shield or safety containment chamber protects individuals from contacting the rotating chambers 402. As the chamber 402 is rotated, pressurized fluid is typically delivered to each chamber 402 via an input feed tube 460, the input cavity 406, the injection orifice 410, the plurality of input apertures 444, the plurality of output apertures 446 and the plurality of injection elements 414. The pressure of the fluid may be monitored using a pressure gauge, similar to 380 in FIG. 25. The injection elements 414 release the pressurized fluid proximate to the interior surface of the internal cavity 418 of the chamber 402 preferably located the furthest distance from the longitudinal axis of the shaft 404.

After the fluid has been released, the fluid flows from the outermost portion of the internal cavity 418 of the chamber 402 inwardly toward the plurality of chamber output apertures 452 located on the interior cavity 418 of the chamber 402 adjacent to the manifold sleeve 422. When the design of the chamber 402 is a triangular toroid, as set forth above, the fluid injected into the chamber 402 decreases in velocity as it moves from the outermost portion of the internal cavity 418 of the chamber 402 inwardly toward the longitudinal axis of the shaft 404. The velocity of the fluid is reduced because the cross-sectional area of the chamber 402 increases in size moving from the outermost portion of the internal cavity 418 of the chamber 402 toward the longitudinal axis of the shaft 404. Injecting the pressurized fluid at the outermost portion of the internal cavity 418 of the chamber 402 positions the fluid so that it must diffuse through the biocatalyst before it leaves the chamber 402 via the plurality of chamber output apertures 452. From these apertures 452, the fluid travels through a chamber output channel 454 to the output aperture 412 and then through the shaft 404 via the outlet cavity 408, where the fluid can be collected from the system 400.

The chamber system 400 positions and incubates a biocatalyst for various beneficial purposes. The chamber system 400 may include a biocatalyst. In another application, the biocatalyst is composed of mammalian cells which are used to produce a variety of beneficial materials including, but not limited to, enzymes. Examples of containment of or immobilization of a biocatalyst are discussed herein and in U.S. Pat. No. 5,821,116.

As mentioned above, the chamber system 400 may include a plurality of chambers 402 located adjacent one another on a single shaft 404. In an embodiment with a plurality of chambers 402, when for instance, mammalian cells, such as hybridoma cells are being utilized to produce monoclonal antibodies; each chamber 402 is capable of immobilizing and maintaining about 2×10¹¹ spherical cells of approximately 20-micron diameter. Conventional stirred tank bioreactors currently used in the production of monoclonal antibodies from similar hybridoma cells would require approximately 100 times the volume of this chamber to maintain a similar number of cells. However, the chamber 402 or plurality of chambers 402 may be increased in diameter, while maintaining their triangular toroidal shape, to immobilize and maintain a larger number of similar sized cells.

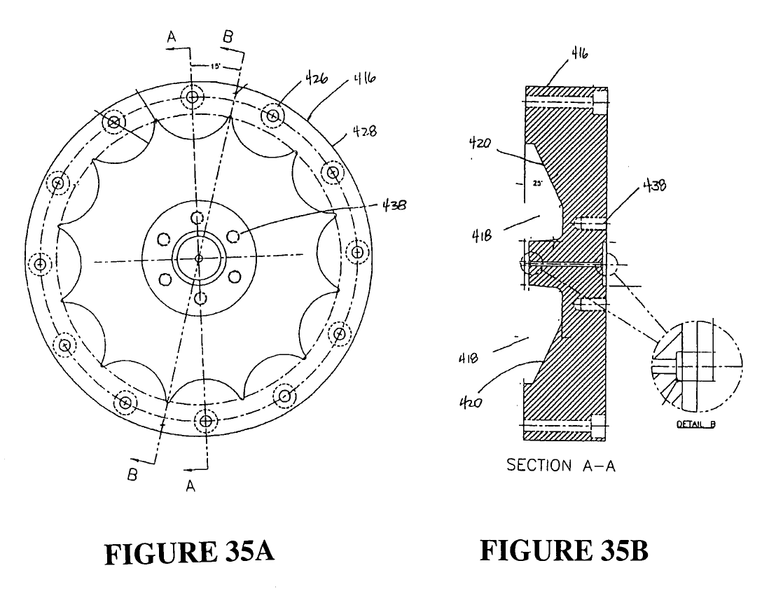

In yet another embodiment of the present invention, a chamber system 500 can have a rotor with a circular-shaped internal cavity 518. As shown in FIGS. 34A and 35A, the internal cavity 418 has a curvilinear-shape with a plurality of curvilinear-shaped walls 419. The internal cavity 418, however, can also be a circular-shaped internal cavity 518 as shown in FIG. 42 or have many other shapes. As with the above discussed embodiments, the chamber system 500 is also directed to an apparatus for substantially immobilizing, containing, suspending and/or incubating a biocatalyst and having at least one chamber 502 for suspending a biocatalyst. In this embodiment, the chamber system 500 includes at least one chamber 502 positioned along a longitudinal axis of a shaft 404. The chamber system 500 may include any number of chambers 502 mounted to the shaft 404. The chamber system 500 is constructed similar to and has similar operational characteristics as the chamber system 400 so for the above discussion also applies to the chamber system 500.

When used in a chamber system 500, two rotor halves are generally coupled together to form a chamber 502. As shown in FIG. 42, a rotor half 505 is depicted having a circular-shaped internal rotor cavity 518. The circular-shaped internal rotor cavity 518 provides a rotor half that does not require curvilinear machining of the rotor cavity 518. The circular-shaped internal rotor cavity 518 reduces manufacturing costs and also provides a scalable chamber system 500 that can be easily scaled for large scale chamber systems. In addition, the rotor halves 505 are circular and are preferably made to have similar configuration which also lowers manufacturing and tooling costs. For use with rotor system 500, one rotor half preferably comprises an exhaust port for nutrients and a circular O-ring channel 532, and the other rotor half may not. In other embodiments, both rotor halves 505 may comprise an input port, exhaust port, or an O-ring channel 532.

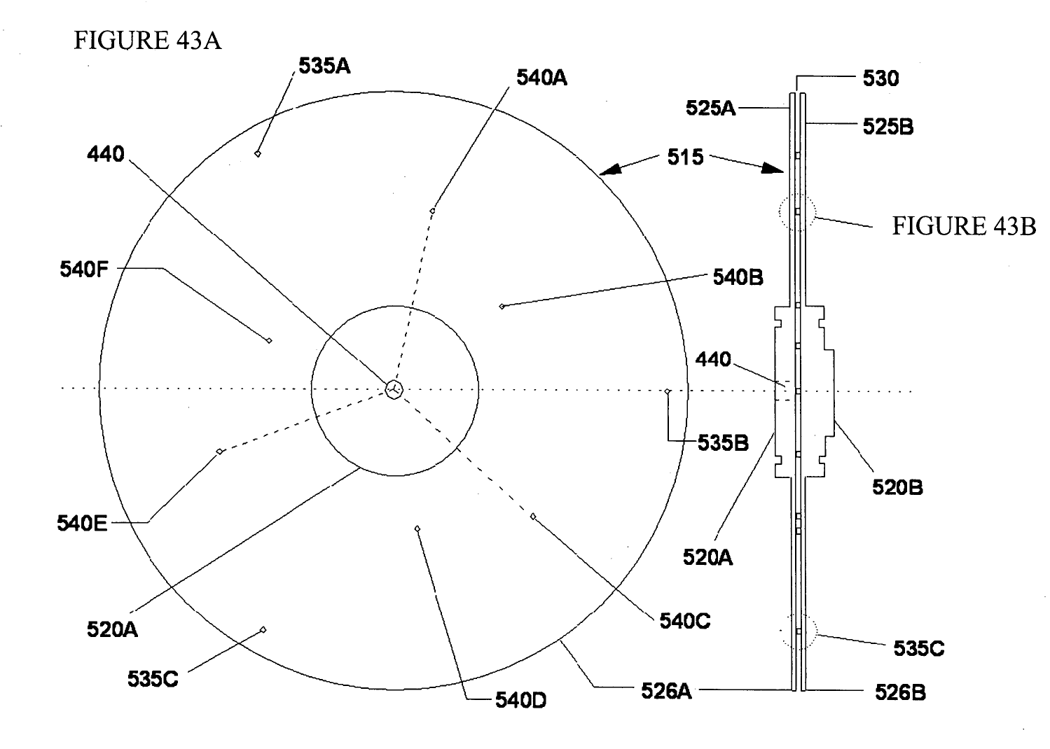

The chamber system 500 can also comprise a flow distributor 515 disposed within the chamber 502. FIGS. 43A-B illustrate a top view and a side view of a flow distributor 515 in accordance with an embodiment of the present invention. As shown, the flow distributor 515 can have a generally circular shape. The circular shape of the flow distributor 515 enables uniformity of liquid flow at all points of the periphery 526 of the flow distributor 515 by virtue of its circular shape and also enables scalability to any desired size.

The flow distributor 515 generally comprises two hub halves 520A-B and two circular discs 525A-B. The two hub halves 520A-B are preferably adapted to engage the rotor halves 505 so that the flow distributor 515 can be suspended within the chamber 502 in the chamber system 500. The circular discs 525A-B can have a thickness of approximately 3/64 inches and have outer peripheries 526A-B. The circular discs 525A-B can have a diameter of approximately 11 cm. In other embodiments, the diameter of the circular discs 525A-B can have a diameter that is very close to the diameter of the internal rotor cavity 518. For example, the diameter of the discs 525A-B can have a ratio in the range of approximately 0.9 to approximately 0.99 relative to the diameter of the internal rotor cavity 518. Configuring the diameter of the circular discs 525A-B close to the diameter of the internal rotor cavity 518 brings the outer peripheries 526A-B close to the circular-shaped internal rotor cavity 518 enabling uniform liquid flow along the outer peripheries 526A-B of the circular discs 525A-B.

The hub halves 520A-B and circular discs 525A-B can be manufactured with various characteristics. The circular discs 525A-B can be attached to the two hub halves 520A-B, such that each one of the circular discs 525A-B is attached to one of the hub halves 520A-B. The circular discs 525A-B can be manufactured with any material that is non-toxic to cultured cells and can withstand centrifugal forces ranging from approximately 1 g to approximately 100 g, where g is the force of gravity. In some embodiments, the discs 525A-B may be made separately from the hub halves 520A-B and attached, adhered, or bonded to the hub halves 520A-B to ensure that the hub halves 520A-B are coupled to the discs 525A-B and to ensure that separation between these components does not occur during system 500 operation. In other embodiments, the hub halves 520A-B and the discs 525A-B can be machined or manufactured as component pieces such that one hub half and one disc are manufactured as one component piece.

The flow distributor 515 can also comprise a fluid conduit 530 defined between the circular discs 525A-B. The fluid conduit 530 enables fluid entering the flow distributor 515 through the injection orifice 410 and the input channel 440 to exit the flow distributor 515 at the peripheries 526A-B of the two circular discs 525A-B. Because the fluid conduit 530 is defined between the discs 525A-B, the flow rate of liquid through the fluid conduit 530 is substantially equal in all radial directions and substantially constant in time over extended time periods. The fluid conduit 530 can be formed by spacing the circular discs 525A-B apart at precise distances with three locating pins 535A-C.

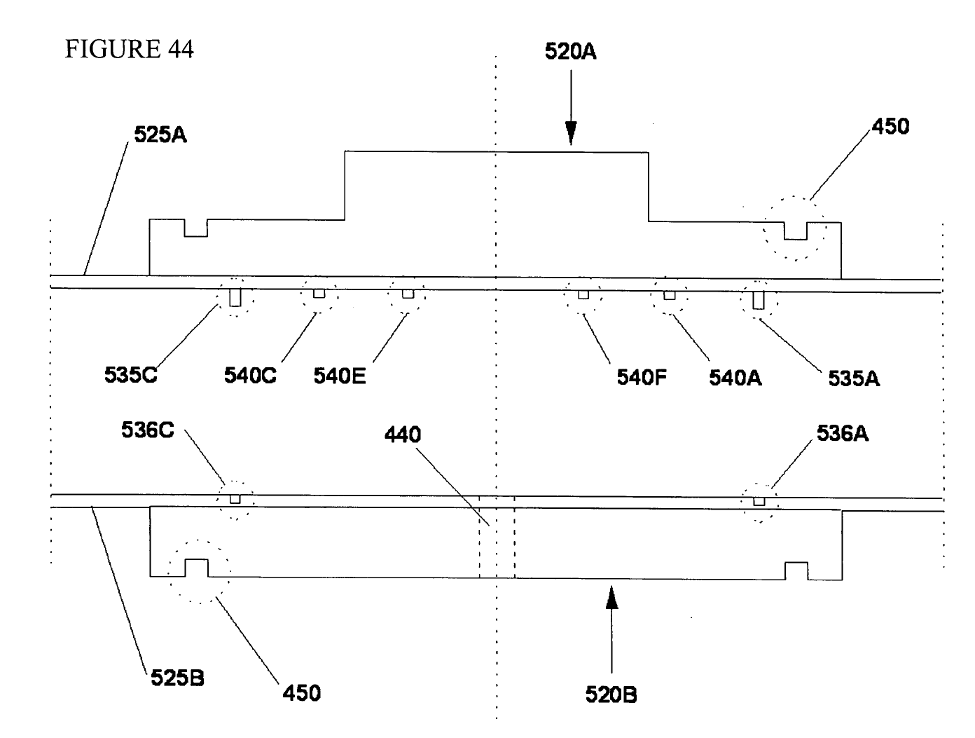

The locating pins 535A-C can be located proximate to the peripheries 526A-B of the circular discs 525A-B. In addition, the three locating pins 535A-C can be arranged so that they are approximately 120 degrees apart from each other. It will be understood that more or less locating pins arranged at different locations may be used in accordance with the various embodiments of the present invention. The locating pins 535A-C can be coupled to circular disc 525A, and the other circular disc 525B can have corresponding apertures 536A-C for receiving the locating pins 535A-C. For example, as shown in FIG. 44, the circular disc 525A can comprise the locating pins 535A and 535C, and the circular disc 525B can define corresponding apertures 536A and 536C for receiving the locating pins 535A and 535C. In some embodiments, the locating pins 535A-C can have a length of approximately 1/16 inches and the apertures 536A-C can have a depth of approximately 1/32 inches. In other embodiments, the locating pins 535A-C can have many other lengths, and the apertures 536A-C can have many other depths. The locating pins 535A-C and corresponding apertures 536A-C can be located approximately 9 cm from the centers of the circular discs 525A-B.

The circular discs 525A-B can also be substantially maintained at predetermined relative positions with one or more spacer stubs. For example, six spacer stubs 540A-F can be used to enable precise separation between the circular discs 525A-B. The spacer stubs 540A-F can be arranged in multiple groups of stubs. For example and as shown in FIG. 43, a first group can comprise stubs 540A, 540C, 540E; and a second group can comprise stubs 540B, 540D, and 540F. The three spacer stubs of each group can be arranged on the circular discs 525A-B so that they are approximately 120 degrees apart from each other. Also, each group of spacer stubs can be rotated out of alignment with the other group and with the triad of locating pins 535A-C such that there is approximately 40 degrees of radial separation between any two pins or stubs, regardless of the triad group to which they belong. This separation minimizes any disturbance in the uniformity of liquid flow at the peripheries 526A-B of the flow distributor 515 due to the passage of liquid past a series of locating pins 535A-C or stubs 540A-F, and enables uniform liquid flow at the outer peripheries 526A-B of the discs 525A-B while maintaining a precise distance between the discs 525A-B to define the fluid conduit 530.

The spacer stubs 540A-F can have various distances from the center of the circular discs 525A-B. For example, the spacer stubs of the first group (540A, 540C, 540E) can be located at approximately seven (7) cm from the center of the circular discs 525A-B, and the spacer stubs of the second group (540B, 540D, 540F) can be located at approximately five (5) cm from the center of the circular discs 525A-B. In addition, it will be understood that more or less spacer stubs arranged at different locations may be used in accordance with the various embodiments of the present invention. In some embodiments, the spacer stubs 540A-F can have a length of 1/32 inches and have a circular cross section. In other embodiments, the spacer stubs 540A-F can have various other lengths and cross sections such that the distance between the discs 525A-B can be modified. For example in some embodiments, as the system 500 is scaled up and liquid flow rates are increased, the distance between the discs 525A-B may be increased to avoid turbulent flow problems of liquid in the fluid conduit 530 defined between the discs 525A-B. Those skilled in the art will understand that increased flow rates of liquid will increase the Reynolds number of the liquid such that the fluid conduit 530 will need to be expanded by increasing the distance between the circular discs 525A-B.

The chamber system 500 comprising a circular-shaped internal rotor cavity 518 and a flow distributor 515 provides numerous advantageous features. First, the rotor body is simpler in its construction thus reducing machining and manufacturing costs. Second, even nutrient flow within the chamber 502 can be accomplished with a single liquid supply pump regardless of the scale (size) of the rotor system 500. Utilizing a single liquid supply pump not only reduces component costs, it also provides a chamber system 500 that is easier to control. In addition, the chamber system 500 comprising a circular-shaped internal rotor cavity 518 and a flow distributor 515 are easily scalable from a small physical size to a large physical size without modifying the design elements of the chamber system 500.

The terms and expressions which have been employed herein are used as terms of description and not of limitation, and there is no intention, in the use of such terms and expressions, of excluding any equivalents of the features shown and described or portions thereof. While various embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all applicable equivalents.

1. An apparatus to contain a biocatalyst comprising:
at least one chamber to immobilize a biocatalyst in which a fluid flows into and out of the chamber, the chamber having an outer portion;
a shaft with a longitudinal axis, the chamber being positioned along the longitudinal axis of the shaft;
a flow distributor mounted within the chamber for evenly distributing fluid flow towards the outer portion of the chamber; and
means for rotating the shaft and the chamber about the longitudinal axis of the shaft.

2. The apparatus of claim 1, the chamber having a substantially circular-shaped outer portion.

3. The apparatus of claim 1, the flow distributor comprising a hub having a fluid entry channel to receive the fluid.

4. The apparatus of claim 1, further comprising a plurality of chambers.

5. The apparatus of claim 1, wherein the chamber has a toroid shape.

6. The apparatus of claim 1, the flow distributor comprising a pair of spaced apart discs each having an outer periphery, the spaced apart discs defining a fluid conduit to provide evenly distributed fluid flow towards the outer peripheries of the spaced apart discs.

7. The apparatus of claim 6, further comprising a spacer disposed between the discs to maintain substantially equidistant separation between the discs.

8. The apparatus of claim 6, wherein one of the discs comprises a locating pin and the other disc defines a corresponding aperture to receive the locating pin such that the pair of spaced apart discs are substantially equidistantly separated.

9. The apparatus of claim 1, further comprising:
an input cavity within the shaft to receive a fluid flow and transmit the fluid flow;
an injection orifice to receive the fluid flow from the input cavity and introduce the fluid flow to the flow distributor;
an output orifice to receive the fluid flow from the chamber; and
an output cavity within the shaft to transfer fluid flow away from the chamber.

10. The apparatus of claim 9, wherein the input cavity and the output cavity extend at least a portion of the length of the shaft.

11. The apparatus of claim 1, wherein the shaft mounts to the exterior of the chamber.

12. The apparatus of claim 1, wherein the flow distributor mounts to the shaft.

13. The apparatus of claim 1, further comprising a sealing means between the shaft and an o-ring that extends around the circumference of the shaft and is adjacent to the flow distributor.

14. The apparatus of claim 1, wherein the longitudinal axis of the shaft is substantially parallel with the surface of the earth.

15. A biocatalyst encapsulation device comprising:
at least one chamber to immobilize a biocatalyst in which a fluid flows into and out of the chamber, the chamber being mounted to a longitudinal axis of a shaft and rotatable about the longitudinal axis of the shaft;
a flow distributor mounted within the chamber for evenly distributing fluid flow towards the outer portion of the chamber, the flow distributor comprising:
a first disc proximate a second disc, the discs defining a fluid conduit between them and having an outer periphery, wherein the fluid is conveyed through the fluid conduit to the outer periphery of the discs; and
a first disc hub and a second disc hub, the first disc hub being coupled to first disc and the second disc hub being coupled to the second disc, wherein at least one of the disc hubs provides the fluid.

17. The device of claim 15, the first disc hub and the second disc hub each comprising O-ring slots to receive an O-ring seal.

18. The device of claim 15, wherein the first disc and the second disc have a thickness of approximately 3/64 inches.

19. The device of claim 15, wherein the first disc is separated from the second disc by approximately 1/16 inches.

20. The device of claim 15, wherein a single fluid pump provides the fluid to the flow distributor.
